(19) United States
(12) Patent Application Publication
(10) Pub. No.: US 2021/0236529 A1
(43) Pub. Date: Aug. 5, 2021

(54) MULTI-TARGETED COMPOSITIONS FOR MITIGATING ACUTE RESPIRATORY DISTRESS SYNDROME

(71) Applicant: Sytheon Ltd, Boonton, NJ (US)

(72) Inventors: Ratan K. Chaudhuri, Lincoln Park, NJ (US); Manpreet Randhawa, Robbinsville, NJ (US)

(73) Assignee: Sytheon Ltd, Boonton, NJ (US)

(21) Appl. No.: 17/224,856

(22) Filed: Apr. 7, 2021

Related U.S. Application Data
(60) Provisional application No. 63/006,501, filed on Apr. 7, 2020.

(51) Int. Cl. A61K 31/7048; A61K 31/7004; A61K 31/513

(52) U.S. Cl. CPC A61K 31/7048; A61K 31/513; A61K 31/7004

(57) ABSTRACT

A method for treatment of viral infections, especially SARS-CoV-2 infections, said method comprising the administration of hydrolysable tannins.

### RELATED APPLICATIONS

The present application claims the benefit of prior U.S. Provisional Patent Application No. 63/006,501, filed Apr. 7, 2020, entitled “Methods and Compositions for Mitigating Symptoms of Acute Respiratory Distress Syndrome,” the contents of which are hereby incorporated herein by reference in their entirety.

### FIELD OF THE INVENTION

The present disclosure relates to the use of hydrolysable tannins for preventing and/or mitigating acute respiratory distress syndrome associated with number of diseases and microbial and viral infections, especially viral infections such as those associated with various influenza and coronaviruses, most especially the SARS-CoV-2 virus, in humans. In particular, the present teaching is directed to compositions and methods using hydrolysable tannins characterized as glucose esterified with gallic-, ellagic-, chebulic-modified ellagic- and modified chebulic-acids and combinations thereof in mitigating and/or preventing the manifestation or occurrence of acute respiratory distress syndrome in individuals infected with an influenza and/or a coronavirus.

### BACKGROUND OF THE INVENTION

Viral infections, especially those associated with influenza and coronavirus, are often widespread and global in nature with varying mortalities. For example, in the 2019-2020 flu season in the US, influenza has manifested a mortality rate of 0.095%; yet, the novel coronavirus, now identified as SARS-CoV-2, which is the cause of COVID-19 and the source of the current pandemic, is already showing at least a 3.4% mortality rate worldwide (httos://www.worldometers.info/coronavirus/coronavirus-death-rate/#who-03-03-20), with much higher levels in certain regions. It remains to be seen what the true number will be on a national as well as a world-wide basis, but it is uncontested that influenza will pale in comparison to the wrath of SARS-CoV-2. Critically important studies emerging from China (Q Ruan et al., Clinical predictors of mortality due to COVID-19 based on an analysis of data of 150 patients from Wuhan, China, Intensive Care Med, https://doi.org/10.1007/s00134-020-05991-x, 2020) suggest that for many patients who die of Covid-19, it may be their own immune system, rather than the virus itself, that deals the fatal blow as a result of a cytokine storm. Identification of compounds that can act on different phases of the viral life cycle or even aid in building and/or supporting the innate immune system can be very useful in managing SARS-CoV-2 infection or reactivation in either immunocompromised individuals or cases of viral drug resistance with nucleoside analogues. The development of a drug with broad-spectrum SARS-CoV-2 inhibitory activity would address this urgent unmet medical need.

COVID-19 has demonstrated itself to make some people much sicker than others: the reason for this is a puzzle that is yet to be solved. Based on recent publications [J Hadjadj et al., Impaired typel interferon activity and inflammatory responses in severe COVID-19 patients, Science, 369(718-724), 2020; M Wadman et al., A rampage through the body, Science, 368(6489):356-360, 2020; M Wadman, Flawed interferon response spurs severe illness, 369(6511):1550-1551, 2020; 369(6500)125-126, 2020], we now know of one very specific predisposing factor: a compromised Type I interferon response. Additionally, it is known that SARS-CoV-2 is also found to be somewhat transparent or difficult to detect even in healthy individuals. Clearly, there is a need for enhanced detection and response within the immune system of individuals who have a compromised Type I interferon response as well as in individuals generally.

Cytokines are essential for orchestrating both innate and adaptive immune responses against microorganisms. Viral defense at mucosal sites depends on interferons (IFN) and IFN stimulated genes (ISGs), either of which may be constitutively expressed to maintain an “antiviral state” (AVS). Interferon regulatory factor 1 (IRF1) plays a critical role in regulating constitutive antiviral gene networks to confer resistance against viral infections in human respiratory epithelial cells. IRF1 prominently participates in antiviral defense by regulating early expression of IFNs and maintaining histone H3K4me1 marks at gene promoter/enhancer regions in homeostatic conditions. In addition to antiviral defense, IRF1 participates in antibacterial defense, autoimmunity, tumor immune surveillance, proinflammatory disease and immune system development, suggesting broad implications for the functional and mechanistic data described recently [D Panda et al., IRF1 Maintains optimal constitutive expression of antiviral genes and regulates the early antiviral response, Frontiers in Immunology, 5 May 2019 https://doi.org/10.3389/fimmu.2019.01019]. IRF1 plays multiple roles toward effective anti-viral responses by maintaining IFN-independent constitutive expression of anti-viral ISGs and supporting early IFN-dependent responses to PRR stimulation.

Interferons (IFNs) are a family of cytokine mediators that are critically involved in alerting the cellular immune system to viral infections of host cells. IFNs not only exhibit important antiviral effects but also exert a key influence on the quality of cellular immune responses and amplify antigen presentation to specific T cells. The three major classes of IFNs are IFN-I, IFN-II, and IFN-III [G Noh, IFN-γ as a major antiviral therapeutic for viral epidemics, including severe acute respiratory syndrome coronavirus (SARS-CoV-2): A clinically forgotten but potential antiviral cytokine and non-virus-specific antiviral as a new antiviral strategy, J Clinical Review & Case Reports, 5(4):217-221, 2020]. Type I IFNs play a critical role in the innate immune response against viral infections. Type II IFN and IFN-gamma have antiviral activity, and type III interferon is also involved in antiviral immunity. IFNs can serve as the first line of immune defense against viral infection. Type I IFNs, of which Interferon alpha (IFN-α) is a member, are secreted by virus-infected cells, while the type II IFN is secreted mainly by T cells, natural killer cells, and macrophages. Type II IFN and IFN-γ are released by immune cells such as cytotoxic T cells and T helper-1 cells.

Type I interferon (IFN-I) response is critical for providing an efficient protection against viral infections. IFN-I production is rapidly triggered by the recognition by host sensors of pathogen-associated molecular patterns (PAMPs), such as viral nucleic acids. IFN-I-induced signaling converges on transcription factors, which rapidly induces the expression of hundreds of genes called interferon-stimulated genes (ISGs) [J W Schoggins, Interferon-Stimulated Genes: What Do They All Do? Annu Rev Virol. 2019; 6(11:567-84. 10.1146/annurev-virology-092818-015756]. This antiviral signaling cascade occurs in virtually all cell types exposed to IFN-l. ISGs, along with other downstream molecules controlled by IFN-I (including proinflammatory cytokines), have diverse functions, ranging from direct inhibition of viral replication to the recruitment and activation of various immune cells [J Crouse et al., Regulation of antiviral T cell responses by type I interferons. Nat Rev Immunol. 2015; 15(4):231-42. 10.1038/nri3806; S Makris et al., Type I Interferons as Regulators of Lung Inflammation. Front Immunol. 2017; 8: 259 10.3389/fimmu.2017.00259]. A robust, well-timed, and localized IFN-I response is thus required as a first line of defense against viral infection because it promotes virus clearance, induces tissue repair, and triggers a prolonged adaptive immune response against viruses [Margarida Sa Ribero et al., Interplay between SARS-CoV-2 and the type I interferon response, PLoS Pathog. 2020 July; 16(7): e1008737]. Despite its criticality, IFN-I response also requires fine-tuning because its overactivation is deleterious to the host.

IFN-I levels in the serum of SARS-Cov-2 infected patients are found to be below the detection levels of commonly used assays. Despite a more efficient replication in human lung tissues, SARS-CoV-2 induced even less IFN-I than SARS-CoV, which is itself a weak inducer in human cells ED Blanco-Melo et al., Imbalanced Host Response to SARS-CoV-2 Drives Development of COVID-19. Cell. 2020; 181(5):1036-45 e9. 10.1016/j.cell.2020.04.026]. An ineffective IFN-I response seems to be a hallmark of other coronavirus infections, as observed with MERS-CoV in ex vivo respiratory tissue cultures. Indeed, coronaviruses have developed multiple strategies to escape and counteract innate sensing and IFN-I production [R W Chan et al., Tropism of and innate immune responses to the novel human betacoronavirus lineage C virus in human ex vivo respiratory organ cultures, J Virol. 2013; 87(12):6604-14. 10.1128/JVI.00009-13]. The delayed IFN-I response indeed promotes the accumulation of pathogenic monocyte-macrophages thus showing negative impact of a delayed IFN-1 response on viral control and disease severity. SARS-CoV encodes at least 10 proteins that allow the virus to either escape or counteract the induction and antiviral action of IFN [Margarida Sa Ribero et al., Interplay between SARS-CoV-2 and the type I interferon response, PLoS Pathog. 2020 July; 16(7): e1008737 and references cited therein]. Initial observations already suggest that the SARS-CoV-2 anti-IFN arsenal is at least as efficient as that of SARS-CoV. Clinical studies showed that coronaviruses evade innate immunity during the first 10 days of infection, which corresponds to a period of widespread inflammation and steadily increasing viral load [J S Peiris et al., Clinical progression and viral load in a community outbreak of coronavirus-associated SARS pneumonia: a prospective study. Lancet. 2003; 361(9371):1767-72. 10.1016/s0140-6736(03)13412-5]. Therefore, there is a need for optimal increase in Type I interferons during the onset of the disease.

Additionally, it is to be appreciated that IFN-γ levels decrease with aging [CJ Yen et al., Age-associated changes in interferon-γ and interleukin 4 secretion by purified human CD4+ and CD8+ T cells, J Biomed Sci, 7:317-321, 2000]. Concurrently, it has been found that susceptibility to and mortality by SARS-CoV-2 is higher in elderly patients. This phenomenon may be related to the relative IFN-γ-deficient status in elderly patients due to ageing. However, it is also found that 1FN-γ gamma production and blood level are also decreased with allergic status; hence, allergic status is believed to result in increased susceptibility to viruses with the allergic condition characterized by a relative IFN-γ deficiency.

SARS-CoV-2 emerged in the human population just over one year ago, yet it seems well adapted to avoid and inhibit the IFN-1 response in its new host. Such efficient strategies allow the virus to replicate and disseminate in infected individuals without encountering the initial host defense. The poor IFN-1 response could explain why viremia peaks at early stages of the disease, at the time of symptoms appearance, and not around 7 to 10 days following symptoms, like during SARS-CoV infections. A recent study suggested that IFNβ may be applicable to improved patient infection status in a combined therapy regiment of IFNβ, lopinavir-ritonavir, and ribavirin [IF Hung et al., Triple combination of interferon beta-1b, lopinavir-ritonavir, and ribavirin in the treatment of patients admitted to hospital with COVID-19: an open-label, randomized, phase 2 trial. Lancet. 2020; 395(10238):1695-704. 10.1016/S0140-6736(20)31042-4]. Additionally, IFN-γ has recently been reported to downregulate the expression of the SARS coronavirus receptor angiotensin-converting enzyme 2 in vitro. [Y Shi et al., Immunopathological characteristics of corona virus disease 2019 cases in Guangzhou, China medRxiv preprint].

Despite their potential, the reality is that interferon therapy, as noted above, has proven less effective than desired. One factor under consideration is whether timing of the treatment is important. Certain new studies suggest interferon treatments may be most helpful in the earliest stages of the disease, but that window oftentimes closes before most people are hospitalized and doctors can treat them, most often before the symptoms and severity of those symptoms rises to the point where medical attention is sought. Indeed, to prevent the overwhelming of emergency rooms and critical care facilities, potential Covid-19 patients are asked to quarantine at home and only seek medical attention if the symptoms are severe. Furthermore, it is also to be appreciated that interferon treatments may also has a lot of side effects, including muscle aches, fever and other ailments associated with flu infections. Even more troublesome is the fact that pitting these virus fighters against the coronavirus too late in the process could actually worsen symptoms, according to animal models cited in a recent review of interferon studies [J Brzoska et al., Interferons in the Therapy of Severe Coronavirus Infections: A Critical Analysis and Recollection of a Forgotten Therapeutic Regimen with Interferon Beta, Drug Res (Stuttg), 70(7):291-297, 2020]. Scientists theorize that the virus's ability to disarm interferons early on may explain other aspects of the disease, such as the out-of-control inflammation reaction that develops in some patients.

A number of virus invasion pathways exist and are continuing to be studied, particularly with respect to the sars-CoV-2 virus. Each of these, as follows, present opportunities for addressing Covid-19.

Angiotensin-Converting Enzyme 2 (ACE2) in SARS-CoV-2 Infection—Throughout the body, the presence of ACE2, which normally helps regulate blood pressure, marks tissues potentially vulnerable to infection, because the virus, particularly the SARS-CoV-2 virus, requires that receptor to enter a cell. Once inside, the virus hijacks the cell's machinery, making myriad copies of itself and invading new cells. However, ACE2 is highly expressed in various organs and tissues such that SARS-CoV-2 not only invades the lungs but also attacks other organs with high ACE2 expression. Furthermore, in addition to the direct viral effects and inflammatory and immune factors, the downregulation of ACE2 and imbalance between the RAS and ACE²/angiotensin-(1-7)/MAS axis may also contribute to the multiple organ injuries in COVID-19. Restoring the balance between the RAS and ACE2/angiotensin-(1-7)/MAS may help attenuate organ injuries in COVID-19 [W Ni et al., Role of angiotensin-converting enzyme 2 (ACE2) in COVID-19, Crit Care, 24: 422, 2020; R K Sharma et al., ACE2 (Angiotensin-Converting Enzyme 2) in cardiopulmonary diseases, Hypertension, 76:651-661, 2020]. The idea that increasing ACE2 would be beneficial is based on the decrease of plasma membrane ACE2 following internalization of SARS-CoV-2 complexed with it; however, an increased ACE2 could also lead to greater cell infection given the strong affinity of the virus for that receptor.

hACE2 (human ACE2): human ACE2 is highly expressed in nasal and airway epithelium, lungs, small intestine, colon, kidneys, and heart with highest expression in intestines [M Gheblawi et al., Angiotensin-converting enzyme 2: SARS-CoV-2 receptor and regulator of the renin-angiotensin system: celebrating the 20th anniversary of the discovery of ACE2, Circ Res, 126:1456-1474, 2020]. The ACE2 expression pattern matters because both SARS-CoV and SARS-CoV-2 use membrane-bound ACE2 as a docking and anchoring site on the surface of epithelial cells [M Hoffmann et al., SARS-CoV-2 cell entry depends on ACE2 and TMPRSS2 and is blocked by a clinically proven protease inhibitor, Cell, 181:271-280, 2020] before viral RNA is internalized into the cytosol of victim cells. The SARS-CoV-2 ACE2-binding domain has a higher affinity for ACE2 versus SARS-CoV [J Shang J et al., Structural basis of receptor recognition by SARS-CoV-2, Nature, 581:221-224, 2020]. The proteolytic cleavage-induced shedding of sACE2 (soluble ACE2) is protective against SARS-CoV-2 virus infection of human epithelial cells in vitro [V Monteil, Kwon H, Prado P, Hagelkrüys A, Wimmer R A, Stahl M, Leopoldi A, Garreta E, Hurtado Del Pozo C, Prosper F, et al., Inhibition of SARS-CoV-2 infections in engineered human tissues using clinical-grade soluble human ACE2, Cell, 181:905-913, 2020]. Therefore, sACE2 may exhibit therapeutic potential to alleviate COVID-19.

ACE2 receptor binding domain on spike protein S1 of SARS-CoV-2 (pdb: 6M17) (RBD) infection: The receptor binding domain (RBD) of the spike subunit S1 of the SARS-CoV-2 virus is the first point of contact between the host and the virus. It plays a key role in the interaction with ACE2 that then lead to the spike subunit S2 domain-mediated membrane fusion and incorporation of viral RNA into host cells [Shekhar et al., Virtual screening and molecular dynamics study of approved drugs as inhibitors of spike protein S1 domain and ACE2 interaction in SARS-CoV-2, J Mol Graph Model, 101:107716, 2020].

Given the nature of the virus, SARS-CoV-2 has been evolving through genetic mutations. There are several covariants arising in different parts of the world. Some of these mutations are happening in the RBD domain of spike protein. One such mutation at the 614th amino-acid position of the spike protein, the amino acid aspartate (D, in biochemical shorthand) was regularly being replaced by glycine (G) because of a copying fault that altered a single nucleotide in the virus's 29,903-letter RNA code also called D614G mutation. The 614 amino acid i.e. Asp614-Gly has been reported to enhance the up conformation of the RBD that makes the virus more infectious, transmissible and susceptible to neutralizing antibodies. It had rapidly become the dominant SARS-CoV-2 lineage in Europe and had then taken hold in the United States, Canada and Australia. Current prophylactic solutions like vaccines are targeted toward RBD domain of virus and with current mutation happening in this specific domain may result in stronger ACE2 binding and poor anti-SARS-CoV mAbs cross-neutralization rendering these vaccines less effective or ineffective (B Korber et al., Tracking Changes in SARS-CoV-2 Spike: Evidence that D614G Increases Infectivity of the COVID-19 Virus. Cell. 2020 Aug. 20; 182(4):812-827.e19. doi: 10.1016/j.cell.2020.06.043 & M Shah et al., Mutations in the SARS-CoV-2 spike RBD are responsible for stronger ACE2 binding and poor anti-SARS-CoV mAbs cross-neutralization, Computational and Structural Biotechnology Journal 2020).

Transmembrane protease/serine subfamily member 2 (TMPRSS2): Transmembrane protease/serine subfamily member 2 (TMPRSS2) is a critical regulator of the plasma membrane ACE2 and is essential for entry of SARS-CoV-2 into cells by priming its spike protein [M Hoffmann et al., SARS-CoV-2 cell entry depends on ACE2 and TMPRSS2 and is blocked by a clinically proven protease inhibitor, Cell, 181:271-280, 2020]. An inhibitor of this enzyme, camostat mesylate, demonstrably reduced SARS entry into cells, [M Hoffmann et al., SARS-CoV-2 cell entry depends on ACE2 and TMPRSS2 and is blocked by a clinically proven protease inhibitor, Cell, 181:271-280, 2020] and a clinical trial is evaluating its efficacy in patients with COVID-19 (https://www.clinicaltrials.gov).

While target pathways present a potentially viable route to addressing viral infections, especially SARS-CoV-2, another potential target is viral RNA replication. Here a number of enzymes and the like have been identified that could be targets for addressing SARS-CoV-2 RNA replication.

3-chymotrypsin-like cysteine protease (3CLPro): 3-chymotrypsin-like cysteine protease (3CLPro), also called main protease (Mpro) of SARS-CoV-2 (pdb: 6w63), controls the copying and manages the life series of SARS-CoV-2. Once the viral genome is inside the host cytoplasm, the ORF1ab fragment of the viral RNA genome is translated into the replicase polyprotein PP1ab which is proteolytically cleaved by the viral enzymes Plpro and 3CLpro (Mpro) to produce 16 non-structural proteins (nsps), including RdRp, and helicase that forms replication-transcription complex [KBK Faheem et al., Druggable targets of SARS-CoV-2 and treatment opportunities for COVID-19, Bioorg Chem, 104:104269, 2020 doi:10.1016/j.bioorg.2020.104269; M A Alamri et al., Structure-based virtual screening and molecular dynamics of phytochemicals derived from Saudi medicinal plants to identify potential COVID-19 therapeutics. Arab J Chem, 13:7224-7234, 2020; doi:10.1016/j.arabjc.2020.08.004].

Papain-like proteases: papain-like proteases (PLpro) is a crucial viral cysteine protease enzyme that cleaves N-terminus of the replicase polyprotein to release several nsps, which includes nsp3 that encoded Plpro [M A Alamri et al., Structure-based virtual screening and molecular dynamics of phytochemicals derived from Saudi medicinal plants to identify potential COVID-19 therapeutics. Arab J Chem, 13:7224-7234, 2020; doi:10.1016/j.arabjc.2020.08.004]. Plpro is implicated not only in the viral replication but also in suppressing the host innate immune response, the latter effect also essential in the virus replication correction because of its nucleic acid-binding domain (NAB) with a nucleic acid chaperon function [MA Alamri et al., Structure-based virtual screening and molecular dynamics of phytochemicals derived from Saudi medicinal plants to identify potential COVID-19 therapeutics. Arab J Chem, 13:7224-7234, 2020; doi: 10. 1016/j.arabjc.2020.08.004].

RNA-dependent RNA polymerase (RdRp): perhaps one of the most important enzymes in viral RNA replication, particularly in SARS-CoV-2 replication is RNA-dependent RNA polymerase (RdRp). SARS-CoV-2 is a positive-strand RNA virus whose replication is mediated by a multi-subunit replication-and-transcription complex of viral nonstructural proteins (nsps) [J. Ziebuhr, The coronavirus replicase, Curr Top Microbiol Immunol, 287: 57-94, 2005]. The core component of this complex is the catalytic subunit (nsp12) of an RNA-dependent RNA polymerase (RdRp) [D G Ahn et al., Biochemical characterization of a recombinant SARS coronavirus nsp12 RNA-dependent RNA polymerase capable of copying viral RNA templates. Arch. Virol, 157:2095-2104, 2012; A J to Velthuis et al., The RNA polymerase activity of SARS-coronavirus nsp12 is primer dependent, Nucleic Acids Res, 38, 203-214, 2010].

By itself, nsp12 has little activity; rather, its functions require accessory factors, including nsp7 and nsp8 [RN Kirchdoerfer and AB Ward, Structure of the SARS-CoV nsp12 polymerase bound to nsp7 and nsp8 co-factors, Nat. Commun, 10:2342, 2019], that increase RdRp template binding and processibility. RdRp has been identified as a potential target of a class of antiviral drugs that are nucleotide analogs; which category includes remdesivir [M Wang et al., Remdesivir and chloroquine effectively inhibit the recently emerged novel coronavirus (2019-nCoV) in vitro, Cell Res, 30:269-271, 2020]. Remdesivir is a prodrug that is converted to the active drug in the triphosphate form [remdesivir triphosphate (RTP)] within cells [D Siegel et al., Discovery and Synthesis of a Phosphoramidate Prodrug of a Pyrrolo[2,1-f][triazin-4-amino] Adenine C-Nucleoside (GS-5734) for the Treatment of Ebola and Emerging Viruses, J Med Chem, 60, 1648-1661, 2017]. However, efforts for the discovery of antiviral drugs to address Covid-19 are hampered because the structures of the SARS-CoV-2 RdRp in complex with an RNA template and with nucleotide inhibitors are not fully understood [W. Yin et al., Structural basis for inhibition of the RNA-dependent RNA polymerase from SARS-CoV-2 by remdesivir, Science, 368(6498):1499-1504, 2020]

In addition to remdesivir, several other nucleotide analog drugs—including favipiravir, ribavirin, galidesivir, and EIDD-2801 have been identified as potential inhibitors of SARS-CoV-2 replication in cell-based assays [T P Sheahan et al., An orally bioavailable broad-spectrum antiviral inhibits SARS-CoV-2 in human airway epithelial cell cultures and multiple coronaviruses in mice, Sci Transl Med, 12, eabb5883, 2020]. Like remdesivir, these nucleotide analogs are thought to inhibit the viral RdRp through nonobligate RNA chain termination, a mechanism that requires conversion of the parent compound to the triphosphate active form [K Warren et al., Protection against filovirus diseases by a novel broad-spectrum nucleoside analogue BCX4430, Nature, 508:402-405, 2014].

Respiratory inflammation, especially acute respiratory distress may arise from any of a number of sources, including environmental exposures, e.g., chemical exposure, smoke, etc., allergens, and, especially, pathogenic microorganism, most especially an influenza virus or a coronavirus, in particular the SARS-CoV and SARS-CoV-2 viruses: the latter the cause of Covid-19. Respiratory distress most typically manifests itself though hyperinflammation of the respiratory system and/or a cytokine storm. As noted above, respiratory distress associated with microorganisms, especially viruses, oftentimes manifests as or in association with a cytokine storm. This is particularly prevalent in influenza and coronavirus infections, especially the latter, and is perhaps the lethal stoke of those afflicted with Covid-19.

Again as noted above, recent research has suggested that high levels of IL-6 and IL-8—two key biomarkers for inflammation and a high-level immune response—is associated with a higher mortality rate in people with community-acquired pneumonia. Severe acute respiratory syndrome (SARS), particularly that which is caused by the SARS coronavirus (SARS-CoV), is a highly communicable disease with the respiratory system, particularly the lungs, as the major pathological target. Although SARS likely stems from overexuberant host inflammatory responses, the exact mechanism leading to the detrimental outcome in patients remains unknown. Pulmonary macrophages (Mφ), airway epithelium, and dendritic cells (DC) are key cellular elements of the host innate defenses against respiratory infections. While pulmonary Mφ are situated at the luminal epithelial surface, DC reside abundantly underneath the epithelium. Such strategic locations of these cells within the airways make it relevant to investigate their likely impact on SARS pathogenesis subsequent to their interaction with infected lung epithelial cells. In the lead-up to the present discovery, a study was conducted to investigate this using highly polarized human lung epithelial Calu-3 cells by using the Transwell culture system. It was found that supernatants harvested from the apical and basolateral domains of infected Calu-3 cells are potent in modulating the intrinsic functions of Mφ and DC, respectively. They prompted the production of cytokines by both Mφ and DC and selectively induced CD40 and CD86 expression only on DC. However, they compromised the abilities of the DC and Mφ in priming naïve T cells and phagocytosis, respectively. Oher researchers have also identified several interleukins, most notably IL-6 and IL-8 as key SARS-CoV-induced epithelial cytokines capable of inhibiting the T-cell-priming ability of DC [T Yoshikawa, et al., Severe Acute Respiratory Syndrome (SARS) Coronavirus-Induced Lung Epithelial Cytokines Exacerbate SARS Pathogenesis by Modulating Intrinsic Functions of Monocyte-Derived Macrophages and Dendritic Cells, J Virology, 83(7):3039-3048, 2009]. Taken together, these results provide insights into the molecular and cellular bases of the host antiviral innate immunity within the lungs that eventually lead to an exacerbated inflammatory cascades and severe tissue damage in SARS patients.

When a virus, particularly a coronavirus, infects a cell, it dumps its genetic payload—a single strand of RNA containing the recipes for making proteins it needs to replicate—into its host. The immune system mobilizes to kill the infected cells before too many copies of the virus can be made. Sometimes, however, that defense mechanism overreacts whereby healthy cells, as well as the sick cells, are killed and a lot of them. Fortunately, most patients do develop their own response against the virus and recover from it, but some patients just have a very brisk response and get really sick.

The lungs constitute a key portal of entry for various respiratory pathogens, and, fortunately, evolution has equipped this vital organ with elaborate host defense systems to maintain its sterility and normal respiratory functions. Epithelium, pulmonary M, and dendritic cells (DC) are three key cellular elements of the airway innate immune system. In addition to functioning as physical and mechanical barriers that separate and eliminate many inhaled materials, lung epithelial cells can directly respond to respiratory infection by secreting various molecules to initiate and sustain cascades of inflammatory responses that ultimately influence the development of adaptive immune responses required to sterilize the infection [LD Martin et al., Airway epithelium as an effector of inflammation: molecular regulation of secondary mediators. Eur. Respir. J. 10:2139-2146, 1997; A J Polito et al., J. Allergy Clin. Immunol, 102:714-718, 1998]. Although this early epithelial response is beneficial in facilitating pathogen clearance, an unregulated and excessive epithelial response can also lead to exacerbated inflammatory responses, causing severe tissue damage [J M Stark et al., Respiratory syncytial virus infection enhances neutrophil and eosinophil adhesion to cultured respiratory epithelial cells. Roles of CD18 and intercellular adhesion molecule-1. J. Immunol. 156:4774-4782, 1996].

During a cytokine storm, an excessive immune response ravages healthy lung tissue, leading to acute respiratory distress and multi-organ failure: untreated, cytokine storm syndrome is usually fatal. Predicting a cytokine storm is difficult, if not impossible, however, in earlier studies, it was found that patients who developed cytokine storm syndrome after viral triggers were subsequently found to have possessed subtle genetic immune defects resulting in the uncontrolled immune response, [GS Schulert et al., Whole-Exome Sequencing Reveals Mutations in Genes Linked to Hemophagocytic Lymphohistiocytosis and Macrophage Activation Syndrome in Fatal Cases of H1N1 Influenza, J Infect Dis, 213(7)1180-1188, 2016].

One common cause of cytokine storms is the over-expression of interleukin-6 (IL-6), one of the most important pro-inflammatory cytokines and one which has been involved in a wide range of disease occurrence and pathogenesis. In two gene therapy clinical trials, the surge of IL-6 was attributed to the cytokine storm and related adverse effects (T Bian et al., Over-expression of Interlukin-6 alone induces dexamethasone-relieved multiple-organ lesion in mice, Immunologic & Host responses in Gene & Cell Therapy, Vol 21, Supplement 1, S173, May 1, 2013, DOl:https://doi.org?10.1016/S1525-0016(16)34884-0). In an animal study, T Bian et al demonstrated that the acute phase symptoms induced by AAV-IL-6 (recombinant adeno-associated virus (MV) vector expressing murine IL-6) were partially prevented and organ damage was alleviated by Dexamethasone: specifically, bone lesions were dramatically recovered and serum paraproteins were largely eliminated. Overall, the results showed that IL-6 alone could potently induce multiple organ inflammatory response, suggesting that IL-6 plays a critical role during the pathological process.

The aforementioned Ruan et al. study also revealed that there was a significant difference in age between the death group and the discharge group (p<0.001) but no difference in the sex ratio (p=0.43). A total of 63% (43/68) of patients in the death group and 41% (34/82) in the discharge group had underlying diseases (p=0.0069). It was also noted that patients with cardiovascular diseases had a significantly increased risk of death when infected with SARS-CoV-2 (p<0.001). The study showed that a total of 16% (11/68) of the patients in the death group had secondary infections, whereas only 1% (1/82) of the patients in the discharge group had secondary infections (p=0.0018). Laboratory results also showed that there were significant differences in white blood cell counts, absolute values of lymphocytes, platelets, albumin, total bilirubin, blood urea nitrogen, blood creatinine, myoglobin, cardiac troponin, C-reactive protein (CRP) and interleukin-6 (IL-6) between the two groups.

An interesting outcome of the response and review of Covid-19 in China was the finding that in some sick patients, viral levels dropped, but levels of IL-6—one of the distress signals used to call the immune system to action—remained high. Hence, the growing belief and concern that cytokine storms and, indeed, acute respiratory distress syndrome, may manifest independently of the progression of the viral infection itself and, instead, arise from over-expression of the immune response. In following, a small study was conducted to test whether Actemra (tocilizumab), a humanized anti-IL-6R monoclonal antibody, would be effective in modulating or interfering with progression of the symptoms of Covid-19. Preliminary findings from a single-arm, 21-patient Chinese trial found that the Covid-19 patients experienced rapidly reduced fevers, with 75% of patients experiencing a reduced need for supplemental oxygen, after treatment with Actemra.

The present Covid-19 pandemic has once again shown the world that it is not ready to deal with the myriad of unknown and/or yet to form viruses, let alone those of which we are aware and their mutations. Despite past instances of Avian flu, SARS as well as the annual influenza viruses, and the massive and ongoing efforts to address them, there are still no effective treatments to mitigate the acute respiratory distress syndrome associated with advance cases. Furthermore, the increasing happenstance of cytokine storms indicate that simply seeking treatments to stop, kill or, at least, slow down the replication and progression of the virus is not sufficient. Rather, efforts must also be directed to addressing and controlling the immunological processes of the patients themselves.

Accordingly, there is a need to identify new and effective treatments and methods for treating individuals suffering from acute respiratory distress syndrome. In particular, there is an urgent and continuing need to identify effective treatments and methods for addressing acute respiratory distress arising from viral infections, particularly influenza viruses and coronaviruses, most especially SARS-CoV-2 virus.

Additionally, there is a need to identify new and effective treatments and methods for preventing or inhibiting the replication of viral RNA and/or for preventing the binding of viruses to their host receptors.

Additionally, there is a need to identify new and effective treatments and methods for activating or promoting the immune response in individuals having a compromised immune response and/or in individuals who have been exposed to and/or infected with viruses that are poorly or unable to be detected by the immune system, particularly with respect to Interferon Type I and Type II, most especially Interferon alpha and/or Interferon gramma.

Additionally, there is a need to identify new and effective treatments and methods for preventing or mitigating the production of excessive pro-inflammatory interleukins, especially IL-6 and/or IL-8, so as to lessen the risk of cytokine storm and/or mitigate the severity thereof.

Finally, there is a need for a method and process by which medical practitioners can tailor the treatment of individuals infected with and/or suffering from viral infections, particularly those associated with and/or know to induce acute respiratory distress syndrome, to more effectively treat the infection based upon the stage or phase of its progression. This need is especially critical with respect to addressing the treatment of individuals infected with an influenza virus or a coronavirus, most especially the SARS-CoV-2 virus.

### SUMMARY

According to a first aspect of the present teaching there is provided a method for preventing, inhibiting, mitigating and/or treating bacterial, fungal and/or viral infections, most especially those associated with or known to cause acute respiratory distress syndrome, most especially for preventing and/or mitigating the manifestation of acute respiratory distress syndrome, said method comprising administering to an individual exposed to or infected with such microorganisms and/or manifesting inflammation of the respiratory system or suffering from acute respiratory distress an effective amount of one or more select hydrolysable tannins, preferably the hydrolysable tannins. In particular, there is provided a method of preventing, inhibiting, mitigating and/or treating acute respiratory distress syndrome associated with or caused by the influenza virus or a coronavirus, most especially the SARS-Cov-2 virus, comprising administering an effective amount of one or more hydrolysable tannins characterized as glucose esterified with gallic-, ellagic-, chebulic-, modified ellagic- and modified chebulic-acids and combinations thereof. The aforementioned hydrolysable tannins may be used as is or are preferably incorporated into a pharmaceutically acceptable carrier for administration to the individual.

According to a second aspect of the present teaching there is provided a method for preventing and/or inhibiting viral RNA replication and/or the binding of viruses, particularly pathogenic viruses, to their host receptor, said method comprising administering to individuals exposed to and/or infected with said viral microorganisms, especially influenza viruses and coronaviruses, most especially the SARS-CoV-2 virus, an effective amount of select hydrolysable tannins, most especially the hydrolysable tannins, said hydrolysable tannins characterized as glucose esterified with gallic-, ellagic-, chebulic-, modified ellagic-, and modified chebulic-acids and combinations thereof. The prevention of viral RNA replication and/or the prevention of the binding of the virus to the host receptor results in a reduced viral load, particularly as compared to an untreated individual, and the prevention, inhibition and/or mitigation of the symptoms association with said viral infections, particularly acute respiratory distress syndrome, most especially hyperinflammation and/or cytokine storm. The aforementioned hydrolysable tannins may be used as is or are preferably incorporated into a pharmaceutically acceptable carrier for administration to the individual.

According to a third aspect of the present teaching there is provided a method for promoting and/or enhancing the immune response in individuals with compromised immune responses and/or to viral infections, particularly infections due to viruses which are known or found to poorly induce or even fail to induce the interferon response, particularly the interferon alpha and interferon gamma responses, most especially the interferon gamma response, especially the coronaviruses such as SARS, SARS-CoV and SARS-CoV-2, most especially SARS-CoV-2. Specifically, it has now been found that interferon alpha and interferon gamma responses may be induced or upregulated by the administration of select hydrolysable tannins, most especially the hydrolysable tannins to the individual exposed to and/or infected with the virus and/or in individuals with a compromised Type I interferon response, wherein the hydrolysable tannins are characterized as glucose esterified with gallic-, ellagic-, chebulic-, modified ellagic- and modified chebulic-acids and combinations thereof.

According to a fourth aspect of the present teaching there is provided a method for preventing, inhibiting, mitigating and or treating acute respiratory distress, most notably, the manifestation of hyperinflammation and or a cytokine storm in the respiratory system, said method comprising administering to individuals exposed to or infected with a virus know to induce or elevate the risk for acute respiratory syndrome an effective amount of select hydrolysable tannins, said hydrolysable tannins being characterized as glucose esterified with gallic-, ellagic-, chebulic-, modified ellagic- and modified chebulic-acids and combinations thereof. Most especially, according to this embodiment, the present method is directed to the administration of said hydrolysable tannins to individuals exposed to and/or infected with influenza viruses and coronaviruses, most especially the SARS-CoV-2 virus.

In each of these embodiments, the hydrolysable tannins may be used alone or in combination with antimicrobial agents, especially antiviral agents (e.g., remdesevir, hydroxychloroquine, etc.), and/or with other therapeutic agents such as plasma treatments, antibody treatments (e.g., Tocilizumab), and the like. The combination treatment is believed synergistic in helping patients recover from acute respiratory distress syndrome, especially from that associated with influenza and coronavirus infections.

According to a fifth aspect of the present teaching there is provided a method for tailoring the treatment of an individual exposed to and/or infected with a virus, particularly viruses which are known or found to poorly induce or even fail to induce the interferon response and/or induce or manifest symptoms of acute respiratory distress syndrome, which method comprises administering to said individual one or more of select hydrolysable tannins, most especially the hydrolysable tannins, the timing, selection of the hydrolysable tannin, and amount of the administration based upon i) the phase of the infection, ii) the viral load, iii) the level of interferon alpha and/or gamma, iv) the level of interleukin 6 and/or 8 and/or v) the manifestation of symptoms of the viral infection, particularly the manifestation of symptoms associated with or a precursor to acute respiratory distress; wherein the hydrolysable tannins are characterized as glucose esterified with gallic-, ellagic-, chebulic-, modified ellagic- and modified chebulic-acids and combinations thereof. This method is especially applicable to the treatment of individuals exposed to and/or infected with an influenza virus or a coronavirus, most especially the SARS-CoV-2 virus, either as an early phase treatment to prevent viral RNA replication and/or binding to its host receptor and/or inducer of the interferon response. Alternatively, it may be used during the course of the infection to prevent, inhibit and/or mitigate symptoms of acute respiratory distress such as hyperinflammation and/or cytokine storms. It may also be used in the late phase of such infections to help bring the immune response back to a more normal, pre-infection, state: thereby addressing hangover or lingering symptoms of the viral infection due to the impact of the infection on the immune and inflammatory responses.

In following, it is very important to understand the disease cycle during its progression, particularly with respect to influenza and coronavirus infections, most especially in the case of Covid-19, as it helps to provide solutions for prevention and cure of the disease. In this regard, viral infections, especially COVID 19, go through four different phases during its disease progression including incubation, early inflammatory, late inflammatory and tail phase: each phase dictated by different pathways. The incubation and early inflammatory phase are primarily driven by the virus itself whereas the autoimmune system dominates the later phase of progression. Finally, the tail phase is characterized by secondary complications including chronic fatigue. Current solutions are targeted toward a specific phase of disease development. For example, some block the virus from entering cells, some delay the immune system response and some block viral replication; however, it is very important to provide the right treatment at the right phase to provide the clinical efficacy. In the case where the selected treatment misses the right opportunity/phase, the disease will progress to the next stage and hence doesn't recover. Corona virus, being asymptomatic during the incubation phase, is very easily missed and cannot be taken care by prophylactic solutions. Hence, there is an urgent and continuing need to develop solutions that can provide the efficacy regardless of the phase of the disease. These solutions are not just taking care of the disease at a particular phase, but also halts the further progression of the disease. According to the present teachings, it has been found that select hydrolysable tannins at the proper levels of administration, is able to address the key physiological markers at essentially all phases of disease progression and, hence, provides a solution to prevent, cure or halt the progression of acute respiratory distress syndrome at any phase.

### DETAILED DESCRIPTION

For a proper understanding and appreciation for the teachings and associated benefits of the compositions and methods taught in this specification, a sense of how viral infections progress, particularly how Covid-19 progresses, is necessary. For convenience, and since it is the key virus of interest, this discussion will be focused on SARS-CoV-2 virus: though, its applicability applies to most if not all viral infections, particularly those arising from an influenza virus or a coronavirus.

While some infectious disease specialists have developed significant experience in treating patients with COVID 19, many therapies continue to be used without evidence of benefit and without regard to timing, potentially causing more harm than would have been realized had no treatment been applied at all. Hence, consensus regarding the phases of COVID-19 is critical for understanding the appropriate timing for the study and delivery of therapeutics. In this respect, it is possible and likely that some of the failures in randomized controlled trials seen to date were due to the mis-timing of treatments. The context of disease phases may explain the failure of remdesivir or monoclonal antibody therapies when given late in disease [Daniel O. Griffin et al., The importance of understanding the Stages of COVID-19 in Treatment and Trials, AIDS Rev, 8:23(1):40-47, 2021; G Lippi et al., Coronavirus disease 2019 (COVID-19): the portrait of a perfect storm, Annals of translational medicine, 8(7):497, 2020]. There are three different phases of Covid 19 progression and the fourth one which is related to imbalance in energy metabolism.

Phase 1: The Pre-Exposure or Incubation Time

The pre-exposure or incubation time is associated with the risk of developing the associated disease as the viral load increases through viral replication. This is the very first phase, starting with the initial exposure or infection and continuing to the first onset or manifestation of severe symptoms and usually lasts between 2 and 11 days (mean incubation time: 6 days), with patients likely to be infectious 1-3 days before the onset of symptoms. Although the true rate of individuals who will remain asymptomatic, or only mildly symptomatic, until terminal viral shedding is still unknown, some evidence suggests that the number could be as high as 50%. Importantly, this rate may be even underestimated due to under-testing or under-reporting. It now seems reasonable to hypothesize that this pre-symptomatic phase is perhaps the most critical for containment of the outbreak. Interestingly, the viral load of asymptomatic, pre-symptomatic, or mildly symptomatic subjects is comparable to that of patients with overt disease. This highlights the significant risk of viral transmission throughout this first phase. Evidence suggests that 50-80% of all cases may be attributed to transmission from an asymptomatic or pre-symptomatic individual. As such, the relatively long incubation time and considerably high rate of asymptomatic-mild symptomatic individuals explains the rise in number of cases despite public health intervention and public awareness. It is during this phase therapies should be more targeted toward inhibiting viral binding and viral replication. It is also the time in which antivirals, monoclonal antibodies and other therapies that augment innate immune responses such as interferons would have the highest potential for benefits versus harm.

Phase 2: Early Inflammatory Phase—Progressive Respiratory Involvement

The viral symptom phase occurs very soon after viral RNA is detectable. For most individuals, that will be the time at which their illness comes to clinical attention. The population in this phase will be predominantly an outpatient population and studying therapeutics in this group will potentially prevent hospitalizations and viral transmission, thereby having a significant impact on resource utilization. The initial clinical manifestations of early inflammatory phase are pulmonary compromise, particularly difficulty or at least the sense of difficulty in breathing, with or without hypoxemia, essentially the early stage of and/or moderate manifestation of acute respiratory distress syndrome, followed by impacts on the cardiac, renal, and other organ systems. Therapeutics that target viral replication and augment the innate immune response such as interferons have a decreasing likelihood of benefit at this later stage of disease since the symptomatic manifestations during this phase are driven by the host's immune responses rather than ongoing viral replication. Here a different type of immunomodulation is required at this stage. The disturbances in the coagulation system also appear to begin during the early inflammatory phase in the 2nd week of illness, but the macrovascular manifestation may not be evident until week three of the illness.

Phase 3: Late Inflammatory Phase—Cytokine Storm

The third phase, which develops in around 15% of all SARS-CoV-2 infected subjects, is perhaps the most challenging and intriguing from a physiopathological perspective. In fact, whilst the respiratory phase is mostly attributable to direct cytopathic lung injury caused by viral replication in pulmonary parenchyma, the late pro-inflammatory phase is instead characterized by an abnormal, almost exaggerated, host reaction against the pathogen, either locally (i.e., in the lung) or systemically, thus mimicking the pathogenesis of severe sepsis and severe inflammatory response syndrome (SIRS). Here the individual is suffering fully developed or server acute respiratory distress. Although the precise mechanisms underlying the onset of this disproportionate host response against the virus remain partially elusive, it has now been acknowledged that SARS-CoV-2 infection of dendritic cells and cells of the monocyte/macrophage lineage triggers their activation and active secretion of a vast array of pro-inflammatory cytokines particularly pro-inflammatory interleukins (ILs) such as IL-6, IL-2, IL-7, and IL-8, monocyte chemoattractant protein 1 (MCP-1), macrophage inflammatory protein 1-α (MIP 1-α), granulocyte colony stimulating factor (GSF), C-X-C motif chemokine 10 (CXCL10) and tumor necrosis factor-α (TNF-α). The renin-angiotensin-aldosterone system (RAAS) also plays a very relevant role in this phase. Specifically, the binding of SARS-CoV-2 to its receptor angiotensin-converting enzyme 2 (ACE2) at the surface of host cells may be associated with profound derangement of RAAS, culminating in the increased activity of angiotensin II (Ang II) and decreased activity of angiotensin 1,7 (Ang 1,7), thus fostering vasoconstrictive, inflammatory, oxidative and fibrotic injuries.

Phase 4: Lack of Energy

The relatively high proportion of people chronically infected with SARS-CoV-2 (‘the long haulers’), who do not make a straight-forward recovery in the post viral period of their illness, almost certainly reflects damage done by the host response to the initial infection. A severe body response such as a cytokine storm can give rise to oxidative and inflammatory damage and generalized oxidative stress, and this suggests that the antioxidant therapies might be beneficial [E Wood et al., Role of mitochondria, oxidative stress and the response to antioxidants in myalgic encephalomyelitis/chronic fatigue syndrome: A possible approach to SARS-CoV-2 ‘long-haulers’? Chronic Dis Trans' Med, 7(1):14-26, 2021]. Antioxidant therapy is known to improve the levels of the abundant natural antioxidant, glutathione (which is important for redox balance), and to strengthen the immune response [ME Soto et al., Is antioxidant therapy a useful complementary measure for Covid-19 treatment? An algorithm for its application, Medicine, 56:1-29, 2020]. Physiological changes in SARS-CoV-2 that enhance the production of reactive oxygen species could be ameliorated by free radical scavengers [G D Mironova et al., Prospects for the use of regulators of oxidative stress in the comprehensive treatment of the novel Coronavirus Disease (COVID-19) and its complications, Eur Rev Med Pharmacol Sci, 24:8585-8591, 2020]. Oxidative stress and ongoing pathogenesis in SARS-CoV-2 are almost certainly linked [L Delgado-Roche et al., Oxidative stress as key player in severe acute respiratory syndrome coronavirus (SARS-CoV) infection, Arch Med Res, 51:384-387, 2020].

The tail phase is now appreciated to be a common feature of COVID 19. Growing numbers of individuals are reporting suffering from this aspect of COVID 19 and support groups have formed of “long haulers”. As we learn more about the post exhaustive fatigue of this disease, it appears to be distinct from that described in chronic fatigue syndrome (CFS). SARS-CoV-2 potentially drains the body's energy and ATP reserves through continued aggressive inflammatory response in the airways, thus damaging the airways and the alveoli, in the lungs, and make carbon dioxide exchange difficult. As a result, the metabolism of the patient cannot provide sufficient energy to support the life processes. Like several other health problems, COVID-19 disease is associated with a difficulty in keeping a balance of the energy budget in the body. As the energy budget worsens, the patient's body tries to balance the budget by scavenging building blocks and energy from the healthy tissues. When the budget becomes unmanageable the patient dies. The disease may be more fatal for the elder patients, both because they may have additional health problems or also because they may have a slower energy metabolism, or their metabolism may produce at a slower rate [M. Özilgen and B Yilmaz, COVID-19 disease causes an energy supply deficit in a patient. Int J Energy Res. 2020; 1-4. 10.1002/er.5883; Bayram Yilmaz et al, Energetic and exergetic costs of COVID-19 infection on the body of a patient, International Journal of Exergy, 32(3): 314-327, 2020].

For purposes of simplicity and a better understanding the present teachings, the following terms have the meanings as presented.

“Preventing” or “prevention” refers to reducing the risk of manifesting acute respiratory distress syndrome.

“Treating” or “treatment” refers to reversing, alleviating, arresting, inhibiting, mitigating or ameliorating at least one of the clinical symptoms associated with acute respiratory distress syndrome, inhibiting the progression of acute respiratory distress syndrome, as well as delaying the onset of at least one or more symptoms of acute respiratory distress syndrome in a patient who has been exposed to or is infected with a microbe, especially a viral agent, that induces or is associated with the manifestation of acute respiratory distress syndrome. In following, treating or treatment also refers to inhibiting acute respiratory distress syndrome, either physically, (e.g., stabilization of a discernible symptom), physiologically, (e.g., stabilization of a physical parameter), or both, and to inhibiting at least one physical parameter that may or may not be discernible to the patient.

“Improve” or “improvement” is used to convey the fact that the present inventive composition has manifested or effected changes, most notably beneficial changes, in either the characteristics and/or the physical attributes of the tissue to which it is being provided, applied or administered, including, for example, boost the Innate and adaptive immunity through interferons (IFNs), reduce replication of coronavirus for example via RNA-dependent RNA polymerase (RdRp) and reduce inflammation by reducing, for example, IL-6 and/or IL-8, etc. These terms are also used to indicate that the symptoms or physical characteristics associated with the diseased state are diminished, reduced or eliminated.

“Inhibiting” generally refers to delaying the onset of the symptoms, delaying or stopping the progression of the symptoms, alleviating the symptoms, or eliminating the symptoms associated with acute respiratory distress syndrome.

Furthermore, again for simplicity, while the present teachings are applicable to addressing or treating acute respiratory distress syndrome generally, whether arising from environmental exposures, e.g., chemical exposure, smoke, etc., allergens, or microorganisms, e.g., fungi, molds, bacteria and viruses, the following description is specifically focused on viral infections, especially influenza and coronavirus infections, most especially exposure and infection by the SARS-CoV-2. Many of the teachings are equally applicable, at least to certain aspects of the acute respiratory distress: though it is also recognized that each type of expose may and does involve different pathways and the development and evolution of acute respiratory distress depending upon the initiator or cause thereof. Similarly, it is recognized that even within the class of viral infections, differences in viral pathways, the development and evolution of the infection, and the disease manifestation are found whereby the exact same response associated with the compositions and methods taught herein may and/or are different.

According to a first aspect of the present teaching there is provided a method for preventing, inhibiting, mitigating and/or treating bacterial, fungal and/or viral infections, most especially those associated with or known to cause acute respiratory distress syndrome, most especially for preventing and/or mitigating the manifestation of acute respiratory distress syndrome, said method comprising administering to an individual exposed to or infected with such microorganisms and/or manifesting inflammation of the respiratory system or suffering from acute respiratory distress an effective amount of one or more select hydrolysable tannins, In particular, there is provided a method of preventing, inhibiting, mitigating and/or treating acute respiratory distress syndrome associated with or caused by the influenza virus or a coronavirus, most especially the SARS-Cov-2 virus, comprising administering an effective amount of one or more hydrolysable tannins characterized as glucose esterified with gallic-, ellagic-, chebulic-, modified ellagic or modified chebulie-acids and combinations thereof.

According to a second aspect of the present teaching there is provided a method for preventing and/or inhibiting viral RNA replication and/or the binding of viruses, particularly pathogenic viruses, to their host receptor, said method comprising administering to individuals exposed to and/or infected with said viral microorganisms, especially influenza viruses and coronaviruses, most especially the SARS-CoV-2 virus, an effective amount of select hydrolysable tannins, most especially the hydrolysable tannins, said hydrolysable tannins characterized as glucose esterified with gallic-, ellagic-, chebulic-, modified ellagic- and modified chebulic acids and combinations thereof. In particular, it has now been found that these hydrolysable tannins interfere with and/or inhibit viral RNA replication and/or the ability of the virus to bind to the ACE2 receptor. The prevention of viral RNA replication and/or the prevention of the binding of the virus to the host receptor results in a reduced viral load, particularly as compared to an untreated individual, and the prevention, inhibition and/or mitigation of the symptoms association with said viral infections, particularly acute respiratory distress syndrome, most especially hyperinflammation and/or cytokine storm.

According to a third aspect of the present teaching there is provided a method for promoting and/or enhancing the immune response in individuals with compromised immune responses and/or to viral infections, particularly infections due to viruses which are known or found to poorly induce or even fail to induce the interferon response, particularly the interferon alpha and interferon gamma responses, most especially the interferon gamma response, especially the coronaviruses such as SARS, SARS-CoV and SARS-CoV-2, most especially SARS-CoV-2. Specifically, it has now been found that the immune response of T cells, NKT cells and/or NK cells and, in particular, the interferon alpha and interferon gamma responses may be induced or upregulated by the administration of select hydrolysable tannins, most especially the hydrolysable tannins to the individual exposed to and/or infected with the virus and/or in individuals with a compromised Type I interferon response, wherein the hydrolysable tannins are characterized as glucose esterified with gallic-, ellagic-, chebulic-, modified ellagic- and modified chebulic-acids and combinations thereof.

According to a fourth aspect of the present teaching there is provided a method for preventing, inhibiting, mitigating and or treating acute respiratory distress, most notably, the manifestation of hyperinflammation and or a cytokine storm in the respiratory system, said method comprising administering to individuals exposed to or infected with a virus know to induce or elevate the risk for acute respiratory syndrome an effective amount of select hydrolysable tannins, most especially the hydrolysable tannins, said hydrolysable tannins characterized as glucose esterified with gallic-, ellagic-, chebulic-, modified ellagic- and modified chebulic-acids and combinations thereof. Most especially, according to this embodiment, the present method is directed to the administration of said hydrolysable tannins to individuals exposed to and/or infected with influenza viruses and coronaviruses, most especially the SARS-CoV-2 virus. In particular, it has been found that the administration of the hydrolysable tannins down-regulate pro-inflammatory interleukins, especially IL-6 and IL-8, as well as other pro-inflammatory cytokines.

According to a fifth aspect of the present teaching there is provided a method for tailoring the treatment of an individual exposed to and/or infected with a virus, particularly viruses which are known or found to poorly induce or even fail to induce the interferon response and/or induce or manifest symptoms of acute respiratory distress syndrome, which method comprises administering to said individual one or more of select hydrolysable tannins, the timing, selection of the hydrolysable tannin, and amount of the administration based upon i) the phase of the infection, ii) the viral load, iii) the level of interferon alpha and/or gamma, iv) the level of interleukin 6 and/or 8 and/or v) the manifestation of symptoms of the viral infection, particularly the manifestation of symptoms associated with or a precursor to acute respiratory distress; wherein the hydrolysable tannins are characterized as glucose esterified with gallic-, ellagic-, chebulic-, modified ellagic- and modified chebulic-acids and combinations thereof. This method is especially applicable to the treatment of individuals exposed to and/or infected with an influenza virus or a coronavirus, most especially the SARS-CoV-2 virus, either as an early phase treatment to prevent viral RNA replication and/or binding to its host receptor and/or inducer of the interferon response. Alternatively, it may be used during the course of the infection to prevent, inhibit and/or mitigate symptoms of acute respiratory distress such as hyperinflammation and/or cytokine storms. It may also be used in the late phase of such infections to help bring the immune response back to a more normal, pre-infection, state: thereby addressing hangover or lingering symptoms of the viral infection due to the impact of the infection on the immune and inflammatory responses.

Hydrolysable tannins (HTs) occur in nature and are generally characterized as glucose esterified with gallic-, ellagic-, chebulic-, modified ellagic- and modified chebulic-acids and combinations thereof. Other hydrolysable tannins, such as those based upon other polyhydric alcohols, e.g., fructose, xylose, saccharose, and structures like hamamelose, as well as those substituted with other acid groups such as hexahydroxydiphenic acid (HHDP) are also believed suitable for use in the practice of the present teaching and are within the scope of its claims, though focus is on the aforementioned hydrolysable tannins. In following, hydrolysable tannins are readily hydrolyzed by acidic, alkali, or enzymatic (tannase or β-glucosidase) hydrolysis, particularly hydrolysis with sulfuric or hydrochloric acid. Polygalloyl esters are called gallotannins (GTs), which give gallic acid (GA) on hydrolysis whereas ellagic esters are referred to ellagitannins (ET) and give hexahydroxydiphenic acid (which spontaneously dehydrates to ellagic acid (EA) upon hydrolysis.

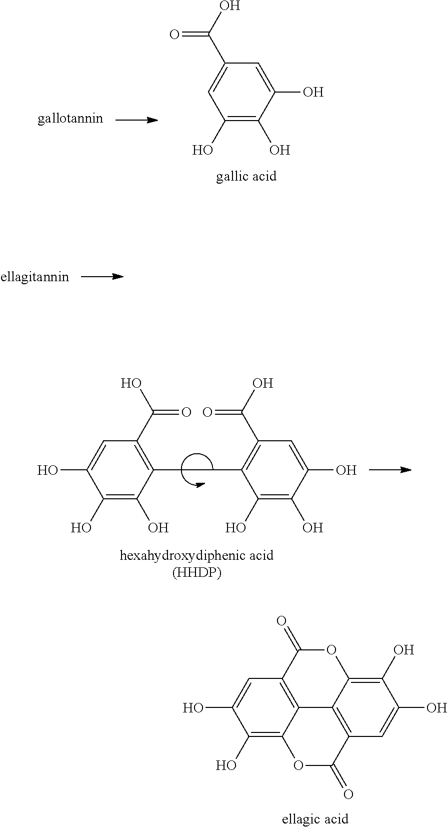 [SMILES: O=C(O)c1cc(O)c(O)c(O)c1 ; O=C(O)c1cc(O)c(O)c(O)c1-c1c(C(=O)O)cc(O)c(O)c1O ; O=c1oc2c(O)c(O)cc3c(=O)oc4c(O)c(O)cc1c4c23]

Hydrolysable tannins may contain both galloyl and hexahydroxydiphenoyl functionalities. ETs can be defined in a narrow sense as hexahydroxydiphenoyl esters of carbohydrates or cyclitols, while the definition of ETs in a wider sense cover compounds derives from further oxidative transformations, including oligomerization processes [T Okuda et al., Ellagitannins Renewed the concepts of tannins, Chapter 1, Chemistry and Biology of Ellagitannins, World Scientific Co Pte: td, http://www.worldscibooks.com/chemistry/679.html]. Monomeric and oligomeric HTs contain one or more polyhydroxyphenoyl groups such as HHDP or its oxidized forms (dehydrohexahydroxydiphenoyl, DHHDP, chebuloyl, or neochebuloyl, m- or p-dehydrodigalloyl and valoneoyl (VL), tergalloyl (TG), macaranoyl, or flavogallonoyl as tris-galloyl and/or gallagyl as tetrakis-galloyl group and so on.

Sources of hydrolysable tannins are well known: preferred hydrolysable tannins are derived from various herbs and plants, including, but not limited to, Occimum gratissmium, Occimum sanctum, Mollugo pentaphylla L, Hypericum triquetrifolium, Ampelopsis brevipedunculata (Maxim.) Trautv. (AB), Withania somnifera, Terminalia chebula, Terminalia bellerica, Terminalia citrina, Terminalia catappa, Euphoria longana, Terminalia macroptera, Terminalia arjuna, Emblica officinalis, Gaila chinensis, teas generally, including Sideritis raseri, particularly from their fruits, leaves, peels and/or roots. Especially preferred sources are the hydrolysable tannins derived from foodstuffs such as, but not limited to, almonds, Terminalia chebula fruit, Terminalia bellerica fruit, Terminalia arjuna fruit, Emblica officinalis fruit, Gaila chinensis fruits, cashew nuts, pistachios, mangos, hazelnuts, persimmons, chestnuts, walnuts, guacas, cloves, pimento, pomegranates, plums, apricots, peaches, bird cherries, strawberries, raspberries, blackberries, black currants, gooseberries, grapes, muscadine grapes, bearberry, and the like. Although natural hydrolysable tannins are preferred, it is also to be appreciated that the hydrolysable tannins for use in the practice of the present teaching can also be synthesized by esterification of polyhydric alcohol with the respective acids, e.g, gallic acid, chebulic acid, ellegic acid and/or HHDP.

As noted above, the selection of the hydrolysable tannin can be tailored to the specific timing of its administration and its intended purpose or objective, all as taught herein. One aspect of the hydrolysable tannins that affects its selection for use at a particular phase of an infection is its hydrophobicity. As shown in the following schematic, hydrophobicity can be selected based upon the groups or acid esters as well as the polyol base:

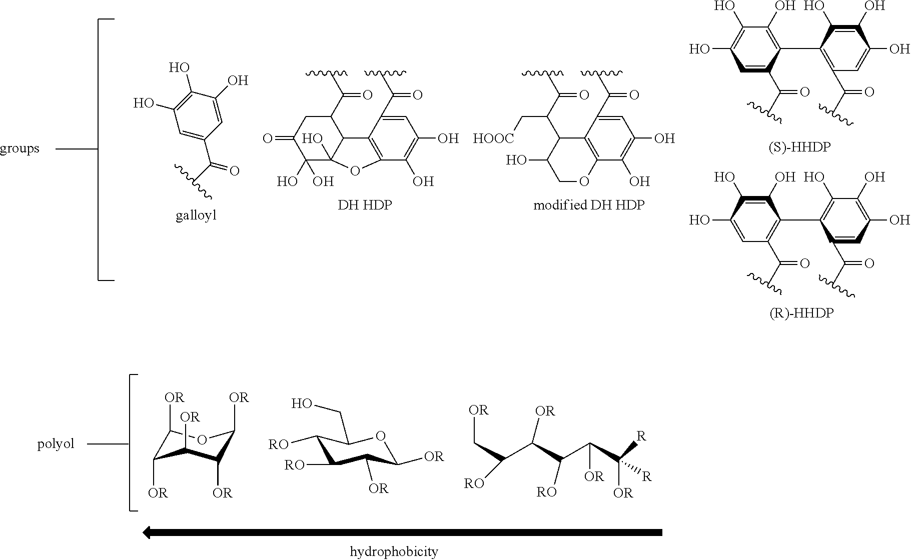

wherein DHHDP=dehydrohexahydroxydiphenoyl, HHDP=hexahydroxydiphenoyl. R can be for instance hydrogen, hydroxyl, galloyl, HHDP or other ET or GT substitute groups [V. Virtanes and M Karonen, Partition Coefficients (IogP) of Hydrolysable Tannins, Molecules, 25(16): 3691, 2020]. In this respect, it has come to be appreciated that hydrophobicity is one of the essential physicochemical properties that affects how a compound interacts with lipids and permeates cell membranes.

Structures of common phenolic acids present as esters in hydrolysable tannins are as follows:

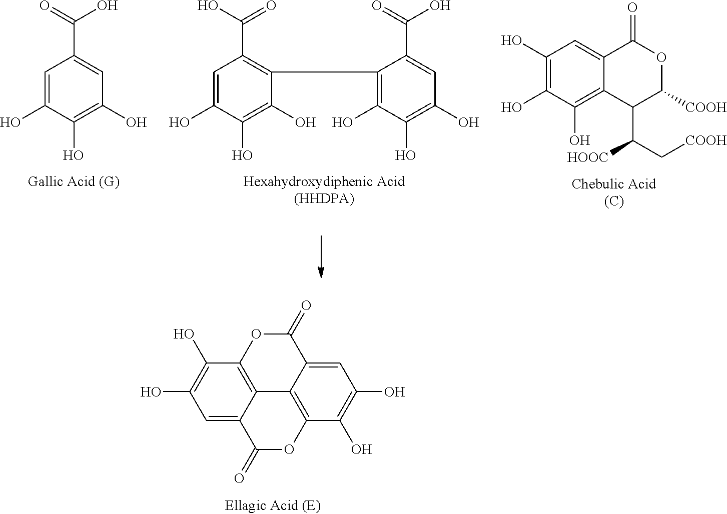 [SMILES: O=C(O)c1cc(O)c(O)c(O)c1 ; O=C(O)c1cc(O)c(O)c(O)c1-c1c(C(=O)O)cc(O)c(O)c1O ; O=C(O)C[C@@H](C(=O)O)C1c2c(cc(O)c(O)c2O)C(=O)O[C@@H]1C(=O)O ; O=c1oc2c(O)c(O)cc3c(=O)oc4c(O)c(O)cc1c4c23]

Preferred hydrolysable tannins for use in the practice of the present teaching include those according to the following structure:

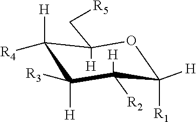

wherein R₁, R₂, R₃, R₄ and R₅, which may be the same or different, are independently selected from hydroxy and ester moieties of gallic acid, ellagic acid, chebulic acid, dehydrohexahydroxydiphenic acid (DHHDP) and hexahydroxydiphenic acid (HHDP), provided that no more than 4, preferably no more than 3, most preferably no more than 2 of the Rx groups are hydroxyl. Exemplary hydrolysable tannins are as follows (“+” means the moiety is bridged across the designated R positions):

Pentagalloyl glucose: R₁=R₂=R₃=R₄=R₅=G

Chebulinic acid: R₁=R₃=R₅=G; R₂+R₄=C

Chebulagic acid: R₁=G; R₂+R₄=C; R₃+R₅=HHDPA

Pedunculagin: R, =OH; R₂+R₃=HHDPA; R₄+R₅=HHDPA

Tellimagrandin I: R₁=OH; R₂=R₃=G; R₄+R₅=HHDPA

Tellimagrandin II: R, =R₂=R₃=G; R₄+R₅=HHDPA

Geraniin: R₁=G; R₂+R₄=DHHDPA; R₃+R₅=HHDPA

Corilagin: R₁=G; R₂=R₄=OH; R₃+R₅=HHDPA

Casuaricitin: R₁=G; R₂+R₃=HHDPA; R₄+R₅=HHDPA

Nupharin A: R₁=R₂=R₅=G; R₃+R₄=HHDPA

The structures of the foregoing as well as a couple additional hydrolysable tannins are as follows:

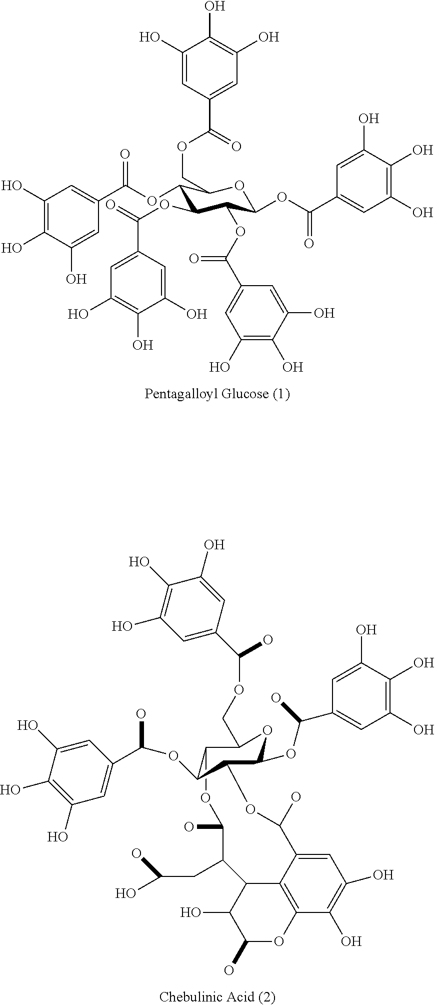

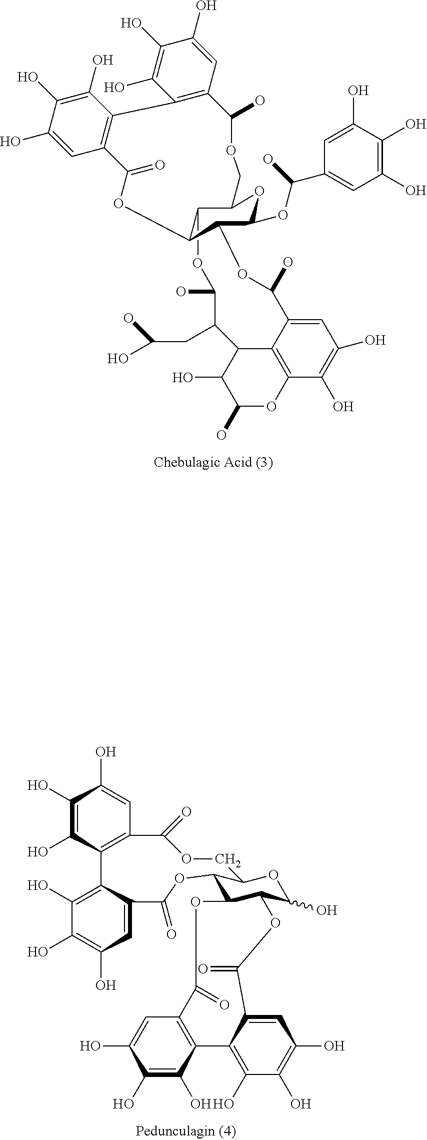

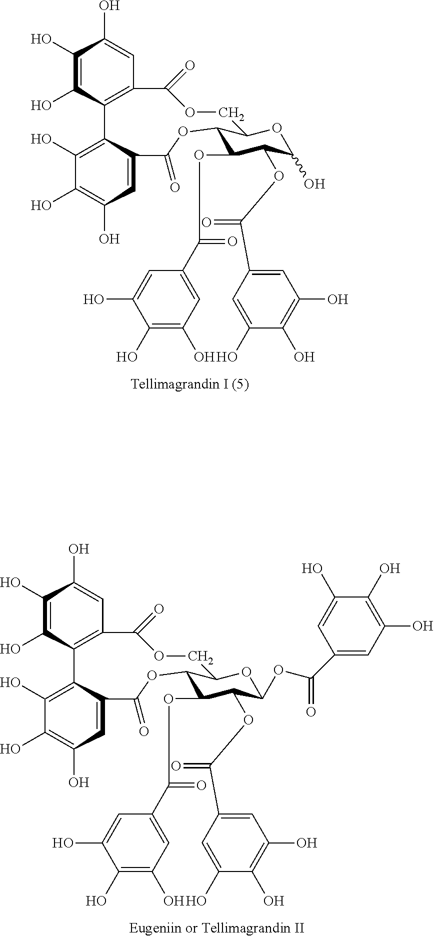

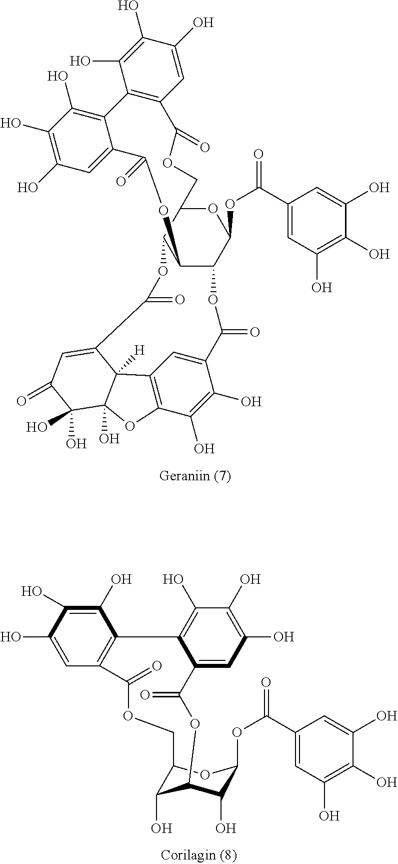

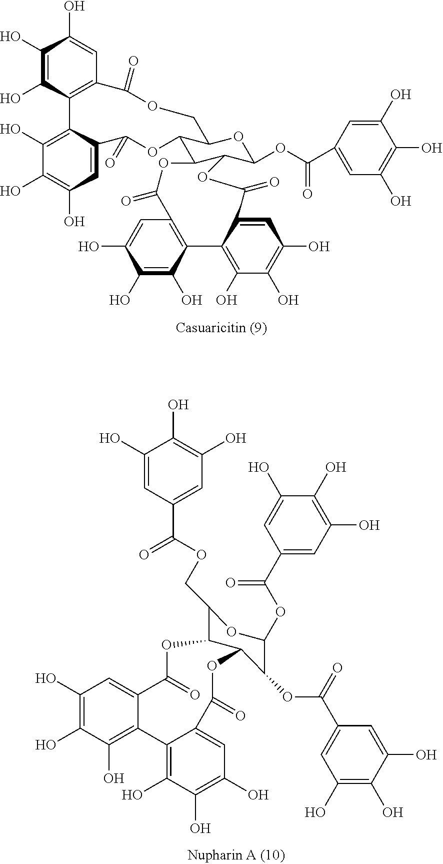

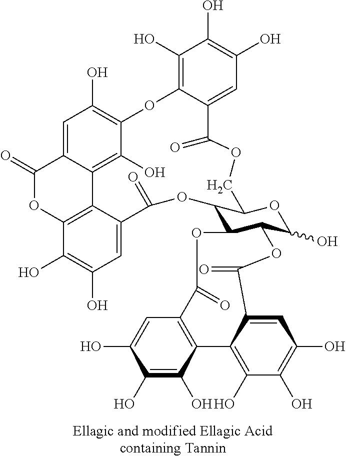

Each of the treatment methods described above involves the administration of an effective amount of the tannins to the individual exposed to or infected with the virus of concern. An “effective amount” is evidenced by the manifestation of an improvement, inhibition, and/or benefit with respect to the purpose for which the hydrolysable tannin is being applied, which, in turn is dependent upon the timing of its administration. For example, an effective amount in relation to the ability to prevent or delay RNA replication and/or the binding of the virus to the host receptor is evidenced by a lower viral load as compared to what is normally expected or common in individuals to whom the hydrolysable tannin was not administered. Similarly, an effective amount for enhancing or initiating the immune response may be established by an up-regulation in the interferon alpha and/or interferon gamma: particularly in individuals whose immune response is compromised. In the case of individuals manifesting the signs of infection, an effective amount is such as will prevent, delay, inhibit and/or improve or shorten the duration of the manifestation of hyperinflammation of the respiratory system and/or cytokine storms. This may manifest visually or may be evaluated by assessing the level of pro-inflammatory cytokines, especially the pro-inflammatory interleukins, such as IL-6 and/or IL8. Preferably, the methods involve the administration of an amount of one or more hydrolysable tannins which effect at least a 20% down regulation in IL-6 and/or IL-8 and/or their corresponding downstream cytokine/chemokine and/or at least a 20% up regulation in IL-12, IFN-alpha and/or IFN-gamma and/or their corresponding downstream cytokine/chemokine as opposed to the response to the same trigger in the absence of the hydrolysable tannin: down regulation and up regulation being evidenced by a reduction or inhibition or a promotion or enhancement, respectively, in the expression or generation/production of the aforementioned interleukins and/or interferons and/or their corresponding downstream cytokine/chemokine, as appropriate. More preferably, the extent of the modulation, i.e., the down regulation and/or up regulation, is at least a 30%, most preferably at least a 50%, as compared to the same trigger in the absence of the hydrolysable tannin.

Following on the foregoing, the specific amount of the hydrolysable tannin to be administered to a given patient will vary depending upon the timing and purpose of its administration, the specific hydrolysable tannin to be administered, the delivery method, the specific disease and/or trigger for the event being addressed (e.g., chemical exposure, bacterial infection, viral infection, etc.), the weight of the patient, etc. The comparative efficacy of the various hydrolysable tannins, as well as combinations thereof, can be ascertained by simple trial and error and/or by further in-vitro assessment of gene expression. Again, administration of the hydrolysable tannins prevent, delay, or mitigate the appearance or manifestation of symptoms of the disease, enable patients to recover faster from acute respiratory distress and/or other manifestations of the immune response being addressed, reduce or lessen the severity of the acute respiratory distress and/or other manifestations of the immune response, and reduce the risk of death from acute respiratory distress, especially from that associated with influenza and coronavirus infections, most especially COVID-19.

The hydrolysable tannins may be administered as a preventative prior to exposure to the pathogen, but, are more likely and preferably administered subsequent to the exposure to the pathogen, but in advance of the manifestation of the symptoms associated with the infection, e.g., following a known exposure, but before diagnostic confirmation. Again, such early administration is difficult absent strict and continual screening tests, hence, the hydrolysable tannins, from a practical perspective, particularly absent clinical symptoms, are more likely to be administered following manifestation of the symptoms of the infection/inflammation. Still, if a person known to exposed also suffers from a compromised immune response and/or there is no indication of a suitable immune response despite the detection of a viral load, it is desirable to administer the hydrolysable tannin as soon as possible to initiate or enhance the immune response, particularly that pertaining to the key interferons, most notably interferon alpha and/or gamma. At the same time, because of the key roles played by cytokines in the immune-response system, it is important not to administer the treatment too early as to interfere with the nature response to the infection or invasion as this may lead to an earlier and faster progression of the disease. For example, it may be desirable to administer the hydrolysable tannins to help initiate and ramp up the immune response; but, to stop the treatment once the immune response is active and allow that response to take its natural path, while continuing to monitor the symptoms and/or level of pro-inflammatory cytokines, particularly the pro-inflammatory interleukins, especially IL-6 and/or IL8. On the other hand, it symptoms worsen or acute respiratory distress syndrome manifests, it is preferable to have initiated administration or, as the case may be, reinitiated, the administration of the hydrolysable tannins once adverse respiratory symptoms are manifesting, particularly that associated with hyperinflammation and/or cytokine storm. The need to administer the hydrolysable tannin is especially warranted if or once other symptoms of the infection or disease are starting to decrease or wane, e.g., if fever is dropping, achiness is less severe, etc, or if viral load is dropping and/or the individual is no longer testing positive, yet, respiratory distress continues as this is indicative of cytokine storm.

The hydrolysable tannins may be administered as is, but are preferably administered as a therapeutic composition in a proper delivery vehicle. Additionally, the hydrolysable tannins may be used alone or in combination with antimicrobial agents, especially antibiotics and/or antiviral agents, and/or with other therapeutic agents such as plasma treatments, antibody treatments (e.g., Tocilizumab), and the like and/or in combination with other anti-inflammatory agents, antioxidants, vitamins and the like. Indeed, it is believed that the afore mentioned combinations are not only cumulative in their benefits but provide synergy in helping patients recover from acute respiratory distress syndrome, especially from that associated with influenza and coronavirus infections. Selection will depend, in part, upon the particular infection or microbe being addressed. For example, indications are that azithromycin, hydroxychloroquine, chloroquine, remdesivir, several nucleotide analog drugs—including favipiravir, ribavirin, galidesivir, and EIDD-2801, and combinations thereof are effective in the treatment of Covid-19. Hence, their combination with the present hydrolysable tannins of the present teachings are beneficial in boosting the Innate and adaptive immunity through interferons (IFNs), reducing replication of coronavirus for example via RNA-dependent RNA polymerase (RdRp) and reducing inflammation by reducing, for example, IL-6 and/or IL-8, etc.

As noted above, hydrolysable tannins can be used as is, i.e., as 100% of the composition to be administered; however, the hydrolysable tannins are preferably incorporated into a pharmaceutical composition in which the hydrolysable tannin(s) account for from about 0.01 to about 99 weight percent of the pharmaceutical composition. Preferably the hydrolysable tannin(s) will comprise from about 0.5 to about 30 wt %, more preferably from about 0.5 to about 20 wt %, most preferably from about 1.0 to about 10 wt % of the pharmaceutical composition. Another factor playing into the concentration of the hydrolysable tannin in the pharmaceutical composition is the dose or rate of application of the composition to the patient. Obviously, dosing itself depends upon a number of factors including the concentration and/or purity of the hydrolysable tannin(s), the individual to whom the composition is to be administered, the mode of administration, the form in which the pharmaceutical composition is to be administered, the disease or symptom to be addressed, etc. Generally speaking, an appropriate dose of the hydrolysable tannin(s), or of the pharmaceutical composition comprising the hydrolysable tannin(s), can be determined according to any one of several well-established protocols including in-vitro and/or in-vivo assays and/or model studies as well as clinical trials. For example, animal studies involving mice, rats, dogs, and/or monkeys can be used to determine an appropriate dose of a pharmaceutical compound. Results from animal studies are typically extrapolated to determine appropriate doses for use in other species, such as for example, humans. Similarly, an appropriate oral dosage for a particular pharmaceutical composition containing one or more hydrolysable tannins will depend, at least in part, on the gastrointestinal absorption properties of the compound, the stability of the compound in the gastrointestinal tract, the pharmacokinetics of the compound and the intended therapeutic profile: all of which is readily ascertainable.

As noted above, the hydrolysable tannins are preferably administered as a therapeutic composition comprising the hydrolysable tannin and a pharmaceutically acceptable vehicle such as a pharmaceutically acceptable diluent, a pharmaceutically acceptable adjuvant, a pharmaceutically acceptable excipient, a pharmaceutically acceptable carrier, or a combination of any of the foregoing with which a pharmacological active agent, including the hydrolysable tannins provided by the present disclosure, can be administered to a patient, which does not destroy or have a marked adverse effect on the activity of the therein contained hydrolysable tannins and which is non-toxic when administered in doses sufficient to provide a therapeutically effective amount. Such vehicles are well known and standard in the pharmacological art. Exemplary carriers include fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The hydrolysable tannin(s), more appropriately, pharmaceutical compositions comprising the hydrolysable tannins, can be administered through any conventional method. The specific mode of application or administration is, in part, dependent upon the form of the pharmaceutical composition, the primary purpose or target of its application (e.g., the application may be oral if intending to address the disease generally or by nasal application or inhalation if intending to address primarily the symptom of acute respiratory distress syndrome. Suitable modes of administration include, for example, intradermal, intramuscular, intraperitoneal, intravenous, subcutaneous, intranasal, epidural, oral, sublingual, intracerebral, intravaginal, transdermal, rectal, nasal or inhalation. The preferred modes of administration are oral, by nasal application, or inhalation. The former allows for absorption through epithelial or mucous linings of the gastrointestinal tract (e.g., oral mucosa, rectal, and intestinal mucosa, etc.) while the latter allows direct application to the tissue of the respiratory tract that is manifesting the symptoms of respiratory distress. Furthermore, again, depending in part upon the form of the administration, the pharmaceutical compositions of the present disclosure can be administered systemically and/or locally. Finally, the form of the pharmaceutical composition containing the hydrolysable tannin(s) and its delivery system varies depending upon the parameters already noted. For example, orally administered pharmaceutical compositions of the present teaching can be in encapsulated form, e.g., encapsulated in liposomes, or as microparticles, microcapsules, capsules, etc.

For preparing pharmaceutical compositions containing the hydrolysable tannin(s) for use in the present methods, pharmaceutically acceptable carriers can be either solid or liquid. Solid form preparations include powders, tablets, pills, capsules, cachets, suppositories, lozenges, and dispersible granules. A solid carrier can be one or more substances which may also act as diluents, flavoring agents, solubilizers, lubricants, suspending agents, binders, preservatives, tablet disintegrating agents, or an encapsulating material including, for example, magnesium carbonate, magnesium state, talc, sugar, lactose, pectin, dextrin, starch, gelatin, tragacanth, chewing gum, methylcellulose, sodium carboxy-methlycellulose, a low melting wax, cocoa butter, and the like. In powders, the carrier is a finely divided solid, which is in a mixture with the finely divided active component. In tablets, the active component is mixed with the carrier having the necessary binding capacity in suitable proportions and compacted in the shape and size desired.

Liquid preparations include solutions, suspensions, and emulsions, for example, water or water-propylene glycol solutions. For example, parenteral injection liquid preparations can be formulated as solutions in aqueous polyethylene glycol solution. The hydrolysable tannin(s) may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral or inhalation use can be prepared by dissolving or suspending the hydrolysable tannin(s) in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxy-methylcellulose, or other well-known suspending agents. Compositions suitable for oral administration in the mouth includes lozenges comprising the active agent in a flavored base, usually sucrose and acacia or tragacanth; pastilles comprising the active ingredient in an inert base such as gelatin and glycerin or sucrose and acacia; and mouthwashes comprising the active ingredient in suitable liquid carrier.

Finally, solutions or suspensions may be applied directly to the nasal cavity by conventional means, for example with a dropper, pipette, or spray. Similarly, solutions or suspensions may be applied directly to the respiratory tract by conventional means, for example, by a spray, nebulizer, or inhaler. The compositions may be provided in single or multi-dose form. In compositions intended for administration to the respiratory tract, including intranasal compositions. The suspension or solutions or active will generally have a small particle size for example of the order of 5 microns or less. Such a particle size may be obtained by means known in the art, for example by micronization, atomization, etc.

Following upon the foregoing, the therapeutic compositions provided by the present disclosure can be formulated in a unit dosage form. A unit dosage form refers to a physically discrete unit suitable as a unitary dose for patients undergoing treatment, with each unit containing a predetermined quantity of the hydrolysable tannin compositions. A unit dosage form can be for a single daily dose, for administration 2 times per day, or one of multiple daily doses, e.g., 3 or more times per day. When multiple daily doses are used, a unit dosage form can be the same or different for each dose. One or more dosage forms typically comprise a dose, which can be administered to a patient at a single point in time or during a time interval.

Of course, one may vary the dosing with time if the desired outcome for the treatment fails to manifest. For example, if viral load increases rapidly or the manifestation of symptoms rapidly advances, particularly in immune compromised individuals or if it appears that a proper or normal immune response is not initiate, it would be desirable to initiate or increase the dosage to promote the immune response. Similarly, if there is a marked worsening of acute respiratory distress, particularly with a subsiding in other factors or symptoms of the disease, it would be appropriate to initiate administration or increased the dose of administration of the hydrolysable tannin(s). For example, one may monitor the status of a patient and adjust the dosage, its frequency, etc. to either drop their levels of pro-inflammatory interleukins to normal levels or to a more controlled, moderate level sufficient to maintain an immune response to the pathogen. Or, if the immune response seems to be lacking, one may want to administer the hydrolysable tannin(s) to boost the innate and adaptive immunity through interferons (IFNs). Furthermore, if exposure is known with viral load increasing, though still asymptomatic, it would be desirable to initiate or increase the dosage to reduce replication of coronavirus via, for example, RNA-dependent RNA polymerase (RdRp) or interference with the binding site of the host. Similarly, it may be desirable to administer a large initial dose to boost the Innate and adaptive immunity through interferons (IFNs) and/or to reduce replication of coronavirus and guard against subsequent hyperinflammation and/or another cytokine storm. Furthermore, one may increase the dose or issue a large dose if the patient's symptoms worsen after treatment has begun.

The compositions containing the hydrolysable tannin(s) (also referred to as the “active” or “actives” hereinafter) can be formulated for immediate release or for delayed or controlled release. In this latter regard, certain embodiments, e.g., an orally administered product, can be adapted for controlled release. Controlled delivery technologies can improve the absorption of an active agent in a particular region, or regions, of the gastrointestinal tract in the case of orally administered doses or in the respiratory tract in the case of nasal or inhalation administered doses. Controlled delivery systems are designed to deliver the active in such a way that its level is maintained within a therapeutically effective window and effective and safe blood levels are maintained for a period as long as the delivery system continues to deliver the active with a particular release profile. Controlled delivery of orally administered actives typically and preferably produces substantially constant blood levels of the active over a period of time as compared to fluctuations observed with immediate release dosage forms. Controlled delivery of inhalation administered actives typically and preferably produces substantially constant levels of the active in the tissue of the respiratory tract over a period of time as compared to fluctuations observed with immediate release dosage forms. For some actives, maintaining a constant blood and/or tissue concentration of the active throughout the course of treatment is the most desirable mode of treatment as immediate release of the active may cause the blood or tissue level of the active to peak above that level required to elicit the most desired response. This results in waste of the active and/or may cause or exacerbate toxic side effects. In contrast, the controlled delivery of the active can result in optimum therapy; not only reducing the frequency of dosing, but also reducing the severity of side effects. Examples of controlled release dosage forms include dissolution-controlled systems, diffusion-controlled systems, ion exchange resins, osmotically controlled systems, erodable matrix systems, pH independent formulations, and gastric retention systems.

An appropriate controlled release oral dosage and ultimate form of a pharmaceutical composition containing the hydrolysable tannin(s) will also depend upon a number of factors. For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Again, it is to be expected that certain hydrolysable tannins are absorbed primarily from the small intestine whereas others are absorbed primarily through the large intestine. It is also to be appreciated that while it is generally accepted that compounds traverse the length of the small intestine in about 3 to 5 hours, there are compounds that are not easily absorbed by the small intestine or that do not dissolve readily. Thus, in these instances, the window for active agent absorption in the small intestine may be too short to provide a desired therapeutic effect in which case large intestinal absorption must be channeled and/or alternate routes of administration pursued.

Where additional pharmacological actives may be and preferably are also present in the compositions according to the present teaching, the amount by which they are present and/or the dosage amount will typically be consistent with their conventional concentration and rates of application. For example, such other actives will be present in an amount of from about 0.5 to about 30 wt. %, more preferably from about 0.5 to about 20 wt. %, most preferably from about 1.0 to about 10 wt. % of the pharmaceutical composition. Of course, as noted, the combination of these other pharmacological actives with the hydrolysable tannin(s) also provide enhanced performance and/or synergy whereby the amounts of each and/or the dose of each is generally less than required for the use of the individual active compounds on their own.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Example 1—Inhibition of RNA-Dependent RNA Polymerase (RdRp)

A study was undertaken in which the impact of select hydrolysable tannins in accordance with the present teaching on the enzymatic activity of SARS-CoV-2 RNA Polymerase (RdRp), in vitro was evaluated. The experiment was performed using the SARS-CoV-2 RNA Polymerase (RdRp) Assay Kit cat. #S2RPA100KE from Profoldin (Hudson, Mass.), according to the manufacturer's instructions provided with kit. The test materials, whose identity and concentration are presented in Table 1, were solubilized and diluted in sterile distilled water and samples were added to the plate according to the plate design. Fluorescent signal proportional to the RNA polymerase activity was quantified at ex/em 485/530 nm with Applied Biosystem, (Foster City, Calif.) multi-well plate fluorometer Cytofluor 4000. Statistical significance was assessed with paired Student test. Deviations of ≥20% as compared to water control with p values below 0.05 were considered statistically significant. Results are summarized in Tables 1 and 2.

TABLE 1
Inhibition of RNA-dependent RNA polymerase (RdRp)

| Test Material | % of Control | p value |
|---|---|---|
| Water (control) | 100 | 1 |
| Chebulinic acid (0.01045 μM) 10 μg | 25 | 0.000 |
| Chebulinic acid (0.0522 μM) 50 μg | 14 | 0.000 |
| Chebulinic acid (0.20905 μM) 100 μg | 7 | 0.000 |
| Chebulagic acid (0.01047 μM) 10 μg | 35 | 0.000 |
| Chebulagic acid (0.05237 μM) 50 μg | 10 | 0.000 |
| Chebulagic acid (0.2094 μM) 100 μg | 8 | 0.000 |
| Chebulic acid (0.02807 μM) 10 μg | 105 | 0.463 |
| Chebulic acid (0.1403 μM) 50 μg | 96 | 0.530 |
| Pentagallolyl glucose (0.01063 μM) 10 μg | 32 | 0.000 |
| Pentagallolyl glucose (0.0531 μM) 50 μg | 12 | 0.000 |
| Pentagallolyl glucose (0.2126 μM) 100 μg | 4 | 0.000 |
| Lopinavir (10 μM) | 91 | 0.237 |
| Lopinavir (50 μM) | 80 | 0.028 |

TABLE 2
EC₅₀ values

| | EC50 | Statistical significance/ |
|---|---|---|
| Test Material | values | Comments |
| Chebulinic acid | <0.01 μM | 0.000/Highly significant |
| Chebulagic acid | <0.01 μM | 0.000/Highly significant |
| Pentagallolyl glucose | <0.01 μM | 0.000/Highly significant |
| Remdesivir* | 0.77 μM | Statistically significant |
| Lopinavir | >50 μM | 0.028/Marginally significant |

*Wang et al., RNA-dependent RNA polymerase of SARS CoV-2 as therapeutic target J Medical Virology, 2021 93:300-310,

As shown in Tables 1 and 2, neither Lopinavir, a commercial inhibitor of viral RNA replication, nor chebulic acid manifested effective inhibition of SARS-CoV-2 RNA replication. In stark contrast, the hydrolysable tannins, namely Chebulinic acid, Chebulagic acid and Pentagallolyl glucose, all exhibited a marked inhibition of SARS-CoV-2 RNA replication. Additionally, each of the hydrolysable tannins demonstrated a markedly lower EC₅₀, even as compared to Remdesivir, currently a key antiviral agent used in the treatment of COVID-19, the disease associated with SARS-CoV-2 infections.

### Example 2—Inhibition of Interleukins

A second study was undertaken to assess the ability of the select hydrolysable tannins to inhibit select pro-inflammatory interleukins, namely IL-6 and IL-8. The study was conducted using reconstituted human ciliated airway tissue. Specifically, Ninety Six 3D ciliated airway tissues (cat. #34839) were obtained from Mattek (Ashland, Mass.) and were cultured in Assay Media (cat. #031921JRD, MatTek). The tissue samples were rinsed twice before incubation with the MTT reagents. The test materials themselves were solubilized and diluted in sterile distilled water and added to the feeder chamber medium contacting the basal side of the tissues on Day 1. The test material was also applied topically on Day 2 together with LPS (added where indicated at 1 μg/ml, from Escherichia coli 0113, Cayman Chemical Company, Ann Arbor, Mich.) for total contact time of 72 h. The experiments were tested in triplicates or duplicates.

Following the exposures to the test materials, 1L-6 and IL-8 were quantified in the tissue culture—conditioned medium by sandwich ELISA (Table 4A and 4B). The effect of the test materials on mitochondrial metabolism was measured by the MTT assay, which quantifies the activity of mitochondrial dehydrogenases, such as succinate dehydrogenase, implicated in the respiratory electron transport chain in mitochondria. The MTT conversion values were also used to standardize the IL-6, IL-8 output data to tissue viability. The results are presented in Tables 3 and 4.

TABLE 3
Inhibition of IL-6

| Test Material | % of Control | p-value |
|---|---|---|
| Water (control) | 100 | 1.000 |
| Chebulinic acid (10 μgm/ml) | 131 | 0.375 |
| Chebulinic acid (50 μgm/ml) | 96 | 0.945 |
| Chebulinic acid (200 μgm/ml) | 18 | 0.029 |
| Chebulagic acid (10 μgm/ml) | 70 | N/A |
| Chebulagic acid (50 μgm/ml) | 40 | 0.098 |
| Chebulagic acid (200 μgm/ml) | 5 | 0.020 |
| Pentagallolyl glucose (10 μgm/ml) | 91 | 0.769 |
| Pentagallolyl glucose (50 μgm/ml) | 64 | 0.201 |
| Pentagallolyl glucose (200 μgm/ml) | 0 | 0.017 |
| Chebulic acid (10 μgm/ml) | 68 | 0.559 |
| Chebulic acid (50 μgm/ml) | 106 | 0.892 |
| Chebulic acid (100 μgm/ml) | 78 | 0.568 |
| Lopinavir (10 μM) | 73 | 0.624 |
| Lopinavir (50 μM) | 81 | 0.572 |

TABLE4
Inhibition of IL-8

| Test Material | % of Control | p value |
|---|---|---|
| Water (control) | 100 | 1.000 |
| Chebulinic acid (10 μg/ml) | 114 | 0.014 |
| Chebulinic acid (50 μg/ml) | 109 | 0.220 |
| Chebulinic acid (200 μg/ml) | 101 | 0.837 |
| Chebulagic acid (10 μg/ml) | 112 | 0.131 |
| Chebulagic acid (50 μg/ml) | 100 | 0.986 |
| Chebulagic acid (200 μg/ml) | 66 | 0.000 |
| Pentagallolyl glucose (10 μg/ml) | 100 | 0.693 |
| Pentagallolyl glucose (50 μgm/ml) | 104 | 0.008 |
| Pentagallolyl glucose (200 μg/ml) | 0 | 0.000 |
| Chebulic acid (10 μg/ml) | 96 | 0.703 |
| Chebulic acid (50 μg/ml) | 108 | 0.131 |
| Chebulic acid (100 μg/ml) | 111 | 0.001 |
| Lopinavir (10 μM) | 103 | 0.667 |
| Lopinavir (50 μM) | 104 | 0.006 |

The results demonstrate an significant inhibition of the pro-inflammatory cytokines IL-6 and IL-8, most notably IL-6, by the select hydrolysable tannins in, what appears as, a dose dependent relationship. This in view of the results of Example 1 above, demonstrates the ability to tailor treatment based upon the dose amount and the timing of its application. Specifically, the lower doses have demonstrated a marked effect on inhibition of viral RNA replication without an adverse effect on the pro-inflammatory cytokines: thereby allowing the immune response to the viral attack that is manifested, to proceed.

### Example 3—Receptor Binding

As noted in the Background, the receptor binding domain on spike protein S1 of SARS-CoV-2 is the first point of contact between the host and the virus and plays a key role in the interaction with ACE2 that then lead to S2 domain-mediated membrane fusion and incorporation of viral RNA into host cells. Accordingly, as study was undertaken to assess the impact of the hydrolysable tannins on the binding of the SARS-CoV-2 at the ACE receptor.

Two docking packages were used to generate docking scores for the SARS-CoV-2. The first is the Vina score from Autodock vina [O Trott and A J Olson, AutoDock Vina: improving the speed and accuracy of docking with a new scoring function, efficient optimization, and multithreading. J Comput Chem 2010; 31:455-618], the second is Glide score from Schrodinger package [R A Friesner et al. Extra precision glide: Docking and scoring incorporating a model of hydrophobic enclosure for protein-ligand complexes, J Med Chem, 49:6177-96, 2006; T A Halgren et al., Glide: A New Approach for Rapid, Accurate Docking and Scoring. 2. Enrichment Factors in Database Screening, J Med Chem, 47:1750-1759, 2000]. The binding scores (presented as Kcal/mole) for the hydrolysable tannins versus other established treatments for Covid-19 are presented in Tables 5A thru 5C.

As evident from the results presented in Tables 5A thru 5C, the select hydrolysable tannins showed significant if not marked lower binding energies (i.e., a tighter binding) as compared to the conventional or currently pursued pharceutical treatments for Covid-19.

TABLE 5A
Binding energy (BE) results for RBD-ACE2

| Test Material | VinaScore | GlideScore |
|---|---|---|
| Chebulagic acid | −7.3 | −4.37 |
| Chebulinic acid | −7.5 | −4.36 |
| Chloroquine | −5.3 | −3.07 |
| Hydroxychloroquine | −5 | −3.71 |
| Dexamethasone | −6.4 | −1.75 |
| Remdesivir | −6.2 | −3.73 |

TABLE 5B
Binding energy results for RdRp

| Test Material | VinaScore | GlideScore |
|---|---|---|
| Chebulagic acid | −10.1 | −6.24 |
| Chebulinic acid | −9.6 | −7.13 |
| Chloroquine | −5.1 | −2.75 |
| Hydroxychloroquine | −5.2 | −3.51 |
| Dexamethasone | −7.6 | −4.44 |
| Remdesivir | −7.0 | −4.62 |

TABLE 5C
Binding energy results for RdRp (cofactor)

| Test Material | VinaScore | GlideScore |
|---|---|---|
| Chebulagic acid | −10.1 | −6.31 |
| Chebulinic acid | −9.7 | −6.91 |
| Chloroquine | −5.3 | −2.68 |
| Hydroxychloroquine | −5.2 | −3.42 |
| Dexamethasone | −7.6 | −4.50 |
| Remdesivir | −6.8 | −4.37 |

### Example 4—Impact on Interferons (IFNα and IFNγ)

A study has been designed, though not yet completed, to assess the impact of the hydrolysable tannins on interferon production and/or activation. In this study, different doses of Chebulinic acid, Chebulagic acid and Pentagalloyl Glucose are being evaluated to demonstrate their efficacy in boosting interferons using Lipopolysaccharide or viral proteins in Human bronchial epithelial cells 3D model system. In this experiment, 3D ciliated airway tissues (cat. #502-3D-24) obtained from Cell Applications (San Diego, Calif.) are cultured in maintenance medium (cat. #511M-3D-100, Cell Applications). The respective test materials are solubilized in sterile water, with and/or without LPS, Syn TC, EMB, and DMSO for the dexamethasone (DEX) control, at 20 mg/ml: all further dilutions are made in sterile distilled water. All test materials, with the exception of LPS, are assayed at different concentrations in μg/ml (LPS is added topically at 1 μg/ml on Day 2). The test materials are added to the feeder chamber medium contacting the basal side of the tissues on Day 1, then also topically treated on Day 2, together with LPS. Following 24 hours of pre-incubation of the tissue samples with the test substances, the tissue samples are then exposed to 1 μg/ml endotoxin LPS and incubation continued for 48 additional hours. At the end of the experiment tissue culture—conditioned medium is stored at −20° C. until further processing. IFN-α and IFN-γ levels are quantified in the tissue culture—conditioned medium by sandwich ELISA. Colorimetric measurements are performed using Molecular Devices microplate reader MAX190 and SoftMax3.1.2PRO software.

Though the current study is yet to be completed, based on the results obtained with extracts of Terminalia chebula, especially high tannin content extracts, as set forth in co-pending U.S. patent application Ser. No. 17/035,405, filed on Sep. 28, 2020, the entire contents of which are incorporated herein by reference, it is expected that the tannins themselves, as now claimed, will demonstrate a marked upregulation of Interferon alpha and Interferon gamma.

Without further elaboration, it is believed that one skilled in the art, using the preceding description, can utilize the present invention to its fullest extent. Furthermore, while the present invention has been described with respect to aforementioned specific embodiments and examples, it should be appreciated that other embodiments, changes and modifications utilizing the concept of the present invention are possible, and within the skill of one in the art, without departing from the spirit and scope of the invention. Furthermore, although not detailed as explicitly above, it is expected that the use of the hydrolysable tannins in the treatment of viral infections, notably, influenza and coronavirus infections, most notably, Covid-19, will help reduce the risk for development of viral drug resistance during therapy, especially with commonly used nucleoside analogues as well as address chronic fatigue associated with the tail phase of COVID 19 disease during the recovery period. In closing, the teachings above, including the preferred specific embodiments, are to be construed as merely illustrative, and not limitative of the remainder of the disclosure and the appended claims in any way whatsoever.

We claim:

1. A method of preventing, inhibiting, mitigating and/or treating Covid-19 comprising administering to an individual exposed to or infected with the SARS-CoV-2 virus an effective amount of one or more hydrolysable tannin.

2. The method of claim 1 wherein the hydrolysable tannins are characterized as glucose esterified with gallic-, ellagic-, chebulic-, modified ellagic-, and modified chebulic-acids and combinations thereof.

3. The method of claim 1 wherein the hydrolysable tannins have the following structure: 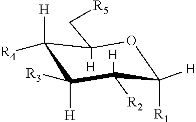
wherein R₁, R₂, R₃, R₄ and R₅, which may be the same or different, are independently selected from hydroxy and ester moieties of gallic acid, ellagic acid, chebulic acid, dehydrohexahydroxydiphenic acid (DHHDP) and hexahydroxydiphenic acid (HHDP), provided that no more than 4, preferably no more than 3, most preferably no more than 2 of the Rₓ groups are hydroxyl.

4. The method of claim 1 wherein the hydrolysable tannin is selected from the group consisting of Pentagalloyl glucose, Chebulinic acid, Chebulagic acid, Pedunculagin, Tellimagrandin I, Tellimagrandin II, Geraniin, Corilagin, Casuaricitin, and Nupharin.

5. The method of claim 1 wherein the hydrolysable tannin is administered following exposure or potential exposure to the SARS-CoV-2 virus but before the manifestation of symptoms of Covid-19 in an amount effective to inhibit or prevent RNA replication and/or prevent or inhibit the binding of the SARS-CoV-2 virus to the receptor site.

6. The method of claim 1 wherein the hydrolysable tannin is administered following exposure or infection to the SARS-CoV-2 virus but before the manifestation of symptoms of Covid-19 in an amount effective to prompt or enhance the initial immune response.

7. The method of claim 6 wherein the hydrolysable tannin is administered to an individual with a compromised immune response.

8. The method of claim 6 wherein the administration of the hydrolysable tannin up-regulates interferon alpha and/or interferon gamma.

9. The method of claim 1 wherein the hydrolysable tannin is administered to an individual manifesting symptoms of Covid-19.

10. The method of claim 9 wherein the hydrolysable tannin is administered to an individual manifesting symptoms of acute respiratory distress syndrome.

11. The method of claim 9 wherein the hydrolysable tannin is administered to an individual manifesting symptoms of cytokine storm and the effective amount is sufficient to down-regulate pro-inflammatory cytokines.

12. The method of claim 11 wherein the effective amount is sufficient to down-regulate Interleukin 6 and/or Interleukin 8.

13. A method of preventing, inhibiting, mitigating and/or treating acute respiratory distress syndrome comprising administering an effective amount of one or more hydrolysable tannins to an individual exposed to a substance or organism known to induce inflammation of the respiratory system and/or suffering a disease or other medical condition known to induce inflammation of the respiratory system.

14. The method of claim 13 wherein the organism is an influenza virus or a corona virus.

15. The method of claim 13 wherein the organism is the SARS-CoV-2 virus.

16. The method of claim 13 wherein the hydrolysable tannins are characterized as glucose esterified with gallic-, ellagic-, chebulic-, modified ellagic-, and modified chebulic-acids and combinations thereof.

17. The method of claim 13 wherein the hydrolysable tannins have the following structure: 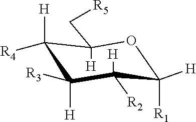
wherein R₁, R₂, R₃, R₄ and R₅, which may be the same or different, are independently selected from hydroxy and ester moieties of gallic acid, ellagic acid, chebulic acid, dehydrohexahydroxydiphenic acid (DHHDP) and hexahydroxydiphenic acid (HHDP), provided that no more than 4, preferably no more than 3, most preferably no more than 2 of the Rₓ groups are hydroxyl.

18. The method of claim 13 wherein the hydrolysable tannin is selected from the group consisting of Pentagalloyl glucose, Chebulinic acid, Chebulagic acid, Pedunculagin, Tellimagrandin I, Tellimagrandin II, Geraniin, Corilagin, Casuaricitin, and Nupharin.

19. The method of claim 13 wherein the hydrolysable tannin is administered following exposure or potential exposure to the substance or organism but before the manifestation of symptoms respiratory distress.

20. The method of claim 13 wherein the hydrolysable tannin is administered following exposure to the substance or organism but before the manifestation of symptoms.

21. The method of claim 13 wherein the hydrolysable tannin is administered to an individual manifesting symptoms of acute respiratory distress syndrome.

22. The method of claim 14 wherein the hydrolysable tannin is administered following exposure or potential exposure to the virus but before the manifestation of symptoms of the associated disease in an amount effective to inhibit or prevent RNA replication and/or prevent or inhibit the binding of the virus to the receptor site.

23. The method of claim 14 wherein the hydrolysable tannin is administered following exposure or infection to the virus but before the manifestation of symptoms of associated disease in an amount effective to prompt or enhance the initial immune response.

24. The method of claim 23 wherein the hydrolysable tannin is administered to an individual with a compromised immune response.

25. The method of claim 23 wherein the administration of the hydrolysable tannin up-regulates interferon alpha and/or interferon gamma.

26. The method of claim 14 wherein the hydrolysable tannin is administered to an individual manifesting symptoms of the associated disease.

27. The method of claim 26 wherein the hydrolysable tannin is administered to an individual manifesting symptoms of acute respiratory distress syndrome.

28. The method of claim 26 wherein the hydrolysable tannin is administered to an individual manifesting symptoms of cytokine storm and the effective amount is sufficient to down-regulate pro-inflammatory cytokines.

29. The method of claim 28 wherein the effective amount is sufficient to down-regulate Interleukin 6 and/or Interleukin 8.

30. A method for tailoring the treatment of an individual exposed to and/or infected with a virus known to cause respiratory distress, which treatment involves the administration of a hydrolysable tannins, said method comprising assessing i) the phase of the infection, ii) the viral load, iii) the level of interferon alpha and/or gamma, iv) the level of interleukin 6 and/or 8 and/or v) the manifestation of symptoms of the viral infection and, based thereon selecting the timing for said administration, the selection of the hydrolysable tannin, and the amount of the administration.
